(19) United States
(12) Patent Application Publication
(10) Pub. No.: US 2022/0193095 A9
(43) Pub. Date: Jun. 23, 2022

(54) PHARMACEUTICAL COMPOSITIONS

(71) Applicant: Shenzhen Pharmacin Co., Ltd., Shenzhen (CN)

(72) Inventors: Zeren Wang, Southbury, CT (US); Shun Chen, Shenzhen (CN)

(21) Appl. No.: 16/815,569

(22) Filed: Mar. 11, 2020

Related U.S. Application Data
(63) Continuation of application No. PCT/CN2020/073099, filed on Jan. 20, 2020.

(30) Foreign Application Priority Data
Jan. 25, 2019 (CN) PCT/CN2019/073112

(51) Int. Cl. A61K 31/58; A61K 47/14; A61K 47/12; A61K 9/48; A61K 9/00

(52) U.S. Cl. CPC A61K 31/58; A61K 47/14; A61K 45/06; A61K 9/48; A61K 9/0053; A61K 47/12

(57) ABSTRACT

Provided are pharmaceutical compositions which comprise a mixture of a lipophilic active pharmaceutical ingredient, at least one long chain fatty acid or at least one fatty acid glyceride, a surfactant, and optionally an antioxidant. Also described are methods for preparing and using such pharmaceutical compositions. The active pharmaceutical ingredient in such a combination has improved bioavailability compared to the active pharmaceutical ingredient alone.

### CROSS-REFERENCE TO RELATED APPLICATION

This application claims the benefit of patent application No. PCT/CN2019/073112, filed on Jan. 25, 2019, the disclosure of which is hereby incorporated by reference in its entirety.

### TECHNICAL FIELD

The present invention belongs to the pharmaceutical field, and specifically relates to pharmaceutical compositions, and their preparation method and use.

### BACKGROUND

Abiraterone, chemical name (3S, 8S, 9S, 10R, 12R, 14S)-10,12-dimethyl-17-(pyridin-3-yl)-2,3,4,7,8,9,10,11,12,13,14,15-dodecahydro-1H-cyclopenta[a]phenanthren-3-ol, and also known as (3β)-17-(3-pyridinyl)-androsta-5,16-dien-3-ol, is an androgen biosynthesis inhibitor, that inhibits 17 α-hydroxylase/C17,20-lyase (CYP17). It is known that abiraterone acetate is rapidly converted in vivo to abiraterone. These structures are depicted below.

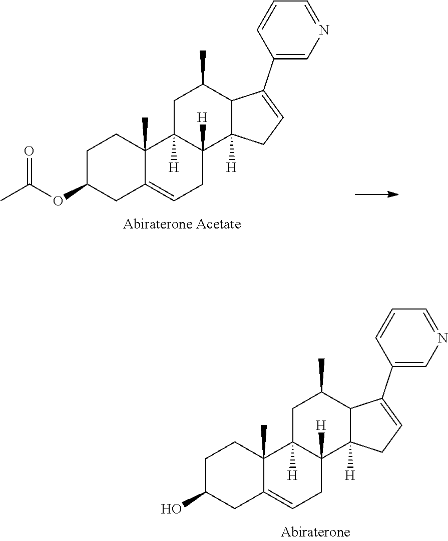

Abiraterone acetate is a lipophilic compound, with a Log P=5.12, making the compound practically insoluble in water. Commercially available abiraterone acetate when administered with meals is known to have the potential to result in increased and highly variable exposures. As a result, the composition must be taken on an empty stomach, either one hour before or two hours after a meal.

There is a need for compositions comprising abiraterone acetate with improved oral bioavailability, permit administering lower doses, that reduce absorption variations caused by food intake, and that reduce in vivo inter-subject absorption variations. More generally, there is a need for improved formulations of highly lipophilic active pharmaceutical ingredients to provide for improved oral bioavailability, permit administering lower doses, that reduce absorption variations caused by food intake, and that reduce in vivo inter-subject absorption variations.

### INCORPORATION BY REFERENCE

All publications and patent applications mentioned in this specification are herein incorporated by reference to the same extent as if each individual publication or patent application was specifically and individually indicated to be incorporated by reference.

### BRIEF SUMMARY

Provided herein are pharmaceutical compositions, comprising: an active pharmaceutical ingredient or a pharmaceutically acceptable salt or ester thereof, wherein the active pharmaceutical ingredient has a calculated log P in octanol-water of >4.0; at least one long chain fatty acid or at least one fatty acid glyceride; and a phospholipid. In some embodiments, the active pharmaceutical ingredient is selected from Table 1 or Table 2. Further provided herein are pharmaceutical compositions wherein the active pharmaceutical ingredient is present in an amount of 10 mg to 1000 mg. Further provided herein are pharmaceutical compositions wherein the active pharmaceutical ingredient is present in an amount of 20 mg to 500 mg. Further provided herein are pharmaceutical compositions wherein the active pharmaceutical ingredient is present in an amount of up to 200 mg. Further provided herein are pharmaceutical compositions wherein the active pharmaceutical ingredient is present in an amount of about 25 mg, about 50 mg, about 100 mg or about 150 mg. Further provided herein are pharmaceutical compositions wherein the active pharmaceutical ingredient comprises up to about 20% of a total weight of the composition. Further provided herein are pharmaceutical compositions wherein the active pharmaceutical ingredient comprises up to about 15% of a total weight of the composition. Further provided herein are pharmaceutical compositions wherein the active pharmaceutical ingredient comprises up to about 10% of a total weight of the composition. Further provided herein are pharmaceutical compositions wherein the active pharmaceutical ingredient comprises up to about 5% of a total weight of the composition. Further provided herein are pharmaceutical compositions wherein the active pharmaceutical ingredient is lipophilic. Further provided herein are pharmaceutical compositions wherein the active pharmaceutical ingredient has a calculated log P of equal or greater than 4.5. Further provided herein are pharmaceutical compositions wherein the active pharmaceutical ingredient has a calculated log P of equal or greater than 5. Further provided herein are pharmaceutical compositions wherein solubility of the active pharmaceutical ingredient in the at least one long chain fatty acid or at least one fatty acid glyceride is at least 25 mg/mL. Further provided herein are pharmaceutical compositions wherein solubility of the active pharmaceutical ingredient in the at least one long chain fatty acid or at least one fatty acid glyceride is at least 50 mg/mL. Further provided herein are pharmaceutical compositions wherein solubility of the active pharmaceutical ingredient in the at least one long chain fatty acid or at least one fatty acid glyceride is at least 100 mg/mL. Further provided herein are pharmaceutical compositions wherein an antioxidant is additionally contained. Further provided herein are pharmaceutical compositions wherein the antioxidant is vitamin E, butylated hydroxytoluene, butylated hydroxyanisole, ascorbyl palmitate, terbuteryl hydroquinone or a combination thereof. Further provided herein are pharmaceutical compositions wherein the antioxidant is butylated hydroxyanisole or butylated hydroxytoluene or a combination thereof. Further provided herein are pharmaceutical compositions wherein the antioxidant is present in an amount less than 1.5% of the total weight of the composition. Further provided herein are pharmaceutical compositions wherein the antioxidant is present in an amount of from about 0.01% to about 1% of the total weight of the composition. Further provided herein are pharmaceutical compositions wherein the pharmaceutical composition is formulated for oral administration. Further provided herein are pharmaceutical compositions wherein the pharmaceutical composition is encapsulated. Further provided herein are pharmaceutical compositions wherein the pharmaceutical composition is encapsulated in a hard capsule. Further provided herein are pharmaceutical compositions wherein the pharmaceutical composition is encapsulated in a soft capsule. Further provided herein are pharmaceutical compositions wherein the pharmaceutical composition is encapsulated in a size 0 or size 00 capsule. Further provided herein are pharmaceutical compositions wherein the pharmaceutical composition is encapsulated in a capsule with a volume of up to 1.5 mL. Further provided herein are pharmaceutical compositions wherein the pharmaceutical composition is encapsulated in a capsule with a volume of from about 0.5 mL to about 1.5 mL. Further provided herein are pharmaceutical compositions wherein the pharmaceutical composition is encapsulated in a capsule with an external diameter of from about 8 mm to about 10 mm. Further provided herein are pharmaceutical compositions wherein the pharmaceutical composition is encapsulated in a capsule of from about 20 mm to about 30 mm in length. Further provided herein are pharmaceutical compositions wherein the pharmaceutical composition is formulated for once daily dosing. Further provided herein are pharmaceutical compositions wherein the pharmaceutical composition is formulated for twice daily dosing.

Provided herein are pharmaceutical compositions comprising: abiraterone or a pharmaceutically acceptable salt thereof or a pharmaceutically acceptable ester thereof; at least one long chain fatty acid or at least one fatty acid glyceride; and a phospholipid. Further provided herein are pharmaceutical compositions comprising abiraterone acetate. Further provided herein are pharmaceutical compositions wherein the at least one long chain fatty acid or the at least one fatty acid glyceride comprises greater than 11 carbon atoms. Further provided herein are pharmaceutical compositions wherein the at least one long chain fatty acid or the at least one fatty acid glyceride comprises greater than 13 carbon atoms. Further provided herein are pharmaceutical compositions wherein the at least one long chain fatty acid or the at least one fatty acid glyceride comprises greater than 15 carbon atoms. Further provided herein are pharmaceutical compositions wherein the at least one long chain fatty acid or the at least one fatty acid glyceride comprises 12, 14, 16, 18, 20, 22 or 24 carbon atoms. Further provided herein are pharmaceutical compositions wherein the at least one long chain fatty acid or the at least one fatty acid glyceride consists of 12, 14, 16, 18, 20, 22 or 24 carbon atoms. Further provided herein are pharmaceutical compositions wherein the at least one long chain fatty acid glyceride comprises one type of fatty acid glyceride. Further provided herein are pharmaceutical compositions wherein the at least one long chain fatty acid glyceride comprise at least two different fatty acid glycerides. Further provided herein are pharmaceutical compositions wherein the at least one long chain fatty acid comprises at least two different long chain fatty acids. Further provided herein are pharmaceutical compositions wherein the at least one long chain fatty acid comprises one type of long chain fatty acid. Further provided herein are pharmaceutical compositions wherein the at least one long chain fatty acid is unsaturated. Further provided herein are pharmaceutical compositions wherein the at least one long chain fatty acid is saturated. Further provided herein are pharmaceutical compositions wherein the at least one long chain fatty acid is Lauric acid, Myristic acid, Palmitic acid, Stearic acid, Arachidic acid, Behenic acid, Lignoceric acid or Cerotic acid. Further provided herein are pharmaceutical compositions wherein the at least one long chain fatty acid is monounsaturated. Further provided herein are pharmaceutical compositions wherein the at least one long chain fatty acid is polyunsaturated. Further provided herein are pharmaceutical compositions wherein the at least one long chain fatty acid is oleic acid, linoleic acid, myristoleic acid, palmitoleic acid, sapienic acid, vaccenic acid, α-linolenic acid or erucic acid. Further provided herein are pharmaceutical compositions wherein the at least one long chain fatty acid is oleic acid. Further provided herein are pharmaceutical compositions wherein the at least one long chain fatty acid is Myristoleic acid, Palmitoleic acid, Sapienic acid, Oleic acid, Elaidic acid, Vaccenic acid, Linoleic acid, Linolelaidic acid, γ-Linolenic acid, α-Linolenic acid, Stearidonic acid, Paullinic acid, Gondoic acid, Dihomo-γ-linolenic acid, Mead acid, Arachidonic acid, Eicosapentaenoic acid, Erucic acid, Docosatetraenoic acid, Docosahexaenoic acid or Nervonic acid. Further provided herein are pharmaceutical compositions wherein the at least one long chain fatty acid or at least one fatty acid glyceride is present in an amount of 100 mg to about 1000 mg. Further provided herein are pharmaceutical compositions wherein the at least one long chain fatty acid or at least one fatty acid glyceride is present in an amount of 250 mg to about 1000 mg. Further provided herein are pharmaceutical compositions wherein the at least one long chain fatty acid or at least one fatty acid glyceride is present in an amount of 500 mg to about 1000 mg. Further provided herein are pharmaceutical compositions wherein the at least one long chain fatty acid or at least one fatty acid glyceride is present in an amount of 600 mg to about 800 mg. Further provided herein are pharmaceutical compositions wherein the at least one long chain fatty acid or at least one fatty acid glyceride is present in an amount of about 700 mg. Further provided herein are pharmaceutical compositions wherein the at least one long chain fatty acid or at least one fatty acid glyceride comprises 30%-95% of the total weight of the composition. Further provided herein are pharmaceutical compositions wherein the at least one long chain fatty acid or at least one fatty acid glyceride comprises 50%-90% of the total weight of the composition. Further provided herein are pharmaceutical compositions wherein the at least one long chain fatty acid or at least one fatty acid glyceride comprises 60%-80% of the total weight of the composition. Further provided herein are pharmaceutical compositions wherein the at least one long chain fatty acid or at least one fatty acid glyceride comprises 70%-80% of the total weight of the composition. Further provided herein are pharmaceutical compositions wherein a ratio of the at least one long chain fatty acid or at least one fatty acid glyceride to phospholipid is greater than 1.5. Further provided herein are pharmaceutical compositions wherein the phospholipid is a component of lecithin. Provided herein are pharmaceutical compositions, comprising: an active pharmaceutical ingredient having a weak base function group or a pharmaceutically acceptable salt thereof, wherein the active pharmaceutical ingredient has a calculated log P in octanol-water equal or greater than 4.0, and a pKa equal or greater than 3.0; at least one long chain fatty acid; and a phospholipid.

Provided herein are pharmaceutical compositions, comprising: abiraterone acetate or a pharmaceutically acceptable salt thereof; oleic acid; and a phospholipid. Further provided herein are pharmaceutical compositions wherein the phospholipid is a phosphatidylcholine, a phosphatidylethanolamine, a phosphatidylinositol, a phosphatidylserine, a plasmalogen, a sphingomyelins, lecithin or phosphatidic acid. Further provided herein are pharmaceutical compositions wherein the phospholipid is a component of lecithin. Further provided herein are pharmaceutical compositions comprising abiraterone acetate or a pharmaceutically acceptable salt thereof; oleic acid; a phospholipid and an antioxidant. Further provided herein are pharmaceutical compositions wherein the antioxidant is vitamin E, butylated hydroxytoluene, butylated hydroxyanisole, ascorbyl palmitate, terbuteryl hydroquinone or a combination thereof. Further provided herein are pharmaceutical compositions wherein the antioxidant is butylated hydroxyanisole or butylated hydroxytoluene or a combination thereof. Further provided herein are pharmaceutical compositions wherein the antioxidant is present in an amount less than 1.5% of the total weight of the composition. Further provided herein are pharmaceutical compositions wherein the antioxidant is present in an amount of from about 0.01% to about 1% of the total weight of the composition. Further provided herein are pharmaceutical compositions wherein the pharmaceutical composition is formulated for oral administration. Further provided herein are pharmaceutical compositions wherein the pharmaceutical composition is encapsulated. Further provided herein are pharmaceutical compositions wherein the pharmaceutical composition is encapsulated in a hard capsule. Further provided herein are pharmaceutical compositions wherein the pharmaceutical composition is encapsulated in a soft capsule. Further provided herein are pharmaceutical compositions wherein the pharmaceutical composition is encapsulated in a size 0 or size 00 capsule. Further provided herein are pharmaceutical compositions wherein the pharmaceutical composition is encapsulated in a capsule with a volume of up to 1.5 mL. Further provided herein are pharmaceutical compositions wherein the pharmaceutical composition is encapsulated in a capsule with a volume of from about 0.5 mL to about 1.5 mL. Further provided herein are pharmaceutical compositions wherein the pharmaceutical composition is encapsulated in a capsule with an external diameter of from about 8 mm to about 10 mm. Further provided herein are pharmaceutical compositions wherein the pharmaceutical composition is encapsulated in a capsule of from about 20 mm to about 30 mm in length. Further provided herein are pharmaceutical compositions wherein the pharmaceutical composition is formulated for once daily dosing. Further provided herein are pharmaceutical compositions wherein the pharmaceutical composition is formulated for twice daily dosing.

Provided herein are pharmaceutical compositions, comprising abiraterone acetate or a pharmaceutically acceptable salt thereof; at least one long chain fatty acid or at least one fatty acid glyceride; and lecithin. Further provided herein are pharmaceutical compositions wherein the lecithin is present in an amount of about 100 mg to about 200 mg. Further provided herein are pharmaceutical compositions wherein the lecithin is present in an amount of about 125 mg to about 175 mg. Further provided herein are pharmaceutical compositions wherein the lecithin is present in an amount of about 150 mg. Further provided herein are pharmaceutical compositions wherein lecithin comprises 10%-20% of the total weight of the composition. Further provided herein are pharmaceutical compositions wherein lecithin comprises 12%-18% of the total weight of the composition. Further provided herein are pharmaceutical compositions wherein lecithin comprises 14%-17% of the total weight of the composition. Further provided herein are pharmaceutical compositions wherein a ratio by weight of the at least one long chain fatty acid or at least one fatty acid glyceride to lecithin is greater than 1.5. Further provided herein are pharmaceutical compositions wherein the lecithin comprises greater than 80% phosphatidylcholine. Further provided herein are pharmaceutical compositions wherein lecithin comprises from 80% to 90% phosphatidylcholine. Further provided herein are pharmaceutical compositions wherein the lecithin is extracted from egg yolk. Further provided herein are pharmaceutical compositions comprising abiraterone acetate or a pharmaceutically acceptable salt thereof; oleic acid; a phospholipid and an antioxidant. Further provided herein are pharmaceutical compositions wherein the antioxidant is vitamin E, butylated hydroxytoluene, butylated hydroxyanisole, ascorbyl palmitate, terbuteryl hydroquinone or a combination thereof. Further provided herein are pharmaceutical compositions wherein the antioxidant is butylated hydroxyanisole or butylated hydroxytoluene or a combination thereof. Further provided herein are pharmaceutical compositions wherein the antioxidant is present in an amount less than 1.5% of the total weight of the composition. Further provided herein are pharmaceutical compositions wherein the antioxidant is present in an amount of from about 0.01% to about 1% of the total weight of the composition. Further provided herein are pharmaceutical compositions wherein the pharmaceutical composition is formulated for oral administration. Further provided herein are pharmaceutical compositions wherein the pharmaceutical composition is encapsulated. Further provided herein are pharmaceutical compositions wherein the pharmaceutical composition is encapsulated in a hard capsule. Further provided herein are pharmaceutical compositions wherein the pharmaceutical composition is encapsulated in a soft capsule. Further provided herein are pharmaceutical compositions wherein the pharmaceutical composition is encapsulated in a size 0 or size 00 capsule. Further provided herein are pharmaceutical compositions wherein the pharmaceutical composition is encapsulated in a capsule with a volume of up to 1.5 mL. Further provided herein are pharmaceutical compositions wherein the pharmaceutical composition is encapsulated in a capsule with a volume of from about 0.5 mL to about 1.5 mL. Further provided herein are pharmaceutical compositions wherein the pharmaceutical composition is encapsulated in a capsule with an external diameter of from about 8 mm to about 10 mm. Further provided herein are pharmaceutical compositions wherein the pharmaceutical composition is encapsulated in a capsule of from about 20 mm to about 30 mm in length. Further provided herein are pharmaceutical compositions wherein the pharmaceutical composition is formulated for once daily dosing. Further provided herein are pharmaceutical compositions wherein the pharmaceutical composition is formulated for twice daily dosing.

Provided herein are pharmaceutical compositions, comprising: abiraterone acetate or a pharmaceutically acceptable salt thereof; oleic acid; lecithin; and optionally, an antioxidant. Further provided herein are pharmaceutical compositions comprising abiraterone acetate. Further provided herein are pharmaceutical compositions wherein abiraterone acetate is present in an amount of from 25 mg to 200 mg. Further provided herein are pharmaceutical compositions wherein abiraterone acetate is present in an amount of about 50 mg, about 100 mg or about 150 mg. Further provided herein are pharmaceutical compositions wherein oleic acid is present in an amount of about 700 mg. Further provided herein are pharmaceutical compositions wherein lecithin is present in an amount of from 100 mg to 200 mg. Further provided herein are pharmaceutical compositions wherein lecithin is present in an amount of about 150 mg. Provided herein are pharmaceutical compositions comprising 25 mg to 200 mg of abiraterone acetate; 600 mg to 800 mg of oleic acid; 100 mg to 200 mg lecithin; and optionally an antioxidant. Further provided herein are pharmaceutical compositions comprising about 50 mg of the abiraterone acetate; about 700 mg of the oleic acid; about 150 mg of the lecithin; and optionally an antioxidant. Further provided herein are pharmaceutical compositions comprising about 100 mg of the abiraterone acetate; about 700 mg of the oleic acid; about 150 mg of the lecithin; and optionally an antioxidant. Further provided herein are pharmaceutical compositions comprising about 150 mg of the abiraterone acetate; about 700 mg of the oleic acid; about 150 mg of the lecithin; and optionally an antioxidant. Further provided herein are pharmaceutical compositions wherein the abiraterone acetate, oleic acid, and lecithin are present in a ratio by weight of from 1:14:3 to 1:4.66:1. Further provided herein are pharmaceutical compositions wherein the abiraterone acetate, oleic acid, and lecithin are present in a ratio by weight of about 1:14:3. Further provided herein are pharmaceutical compositions wherein the abiraterone acetate, oleic acid, and lecithin are present in a ratio by weight of about 1:7:1.5. Further provided herein are pharmaceutical compositions wherein the abiraterone acetate, oleic acid, and lecithin are present in a ratio by weight of about 1:4.66:1. Further provided herein are pharmaceutical compositions wherein the antioxidant is vitamin E, butylated hydroxytoluene, butylated hydroxyanisole, ascorbyl palmitate, terbuteryl hydroquinone or a combination thereof. Further provided herein are pharmaceutical compositions wherein the antioxidant is butylated hydroxyanisole or butylated hydroxytoluene or a combination thereof. Further provided herein are pharmaceutical compositions wherein the antioxidant is present in an amount less than 1.5% of the total weight of the composition. Further provided herein are pharmaceutical compositions wherein the antioxidant is present in an amount of from about 0.01% to about 1% of the total weight of the composition. Further provided herein are pharmaceutical compositions wherein the pharmaceutical composition is formulated for oral administration. Further provided herein are pharmaceutical compositions wherein the pharmaceutical composition is encapsulated. Further provided herein are pharmaceutical compositions wherein the pharmaceutical composition is encapsulated in a hard capsule. Further provided herein are pharmaceutical compositions wherein the pharmaceutical composition is encapsulated in a soft capsule. Further provided herein are pharmaceutical compositions wherein the pharmaceutical composition is encapsulated in a size 0 or size 00 capsule. Further provided herein are pharmaceutical compositions wherein the pharmaceutical composition is encapsulated in a capsule with a volume of up to 1.5 mL. Further provided herein are pharmaceutical compositions wherein the pharmaceutical composition is encapsulated in a capsule with a volume of from about 0.5 mL to about 1.5 mL. Further provided herein are pharmaceutical compositions wherein the pharmaceutical composition is encapsulated in a capsule with an external diameter of from about 8 mm to about 10 mm. Further provided herein are pharmaceutical compositions wherein the pharmaceutical composition is encapsulated in a capsule of from about 20 mm to about 30 mm in length. Further provided herein are pharmaceutical compositions wherein the pharmaceutical composition is formulated for once daily dosing. Further provided herein are pharmaceutical compositions wherein the pharmaceutical composition is formulated for twice daily dosing.

Provided herein are methods for modulating androgen receptor activity, comprising administering to a subject in need of modulating androgen receptor activity, a pharmaceutical composition described herein. In some embodiments, the pharmaceutical composition comprises an active pharmaceutical ingredient selected from Table 1 or Table 2. Provided herein are methods for treating prostate cancer, breast cancer, ovarian cancer, endometrial cancer, bladder cancer, pancreatic cancer, hepatocellular cancer, kidney cancer, liver cancer, salivary gland carcinoma, hair loss, acne, hirsutism, ovarian cysts, polycystic ovary disease, precocious puberty, spinal and bulbar muscular atrophy, or age-related macular degeneration, comprising administering to a subject in need thereof the pharmaceutical composition described herein. Provided herein are methods for treating prostate cancer, comprising administering to a subject in need thereof the pharmaceutical composition described herein. Further provided herein are methods wherein the prostate cancer is selected from one or more of the following: castration-resistant prostate cancer; metastatic castration-resistant prostate cancer; castration-recurrent prostate cancer; high-risk castration-sensitive prostate cancer; metastatic high-risk castration-sensitive prostate cancer; hormone-resistant prostate cancer; hormone-refractory prostate cancer; androgen-independent prostate cancer; androgen deprivation resistant prostate cancer; androgen ablation resistant prostate cancer; androgen depletion-independent prostate cancer; anti-androgen-recurrent prostate cancer; metastatic castration-resistant prostate cancer in patients who have already received prior chemotherapy containing docetaxel; newly diagnosed high risk metastatic hormone sensitive prostate cancer (mHSPC); metastatic castration resistant prostate cancer in patients who are asymptomatic or mildly symptomatic after failure of androgen deprivation therapy in whom chemotherapy is not yet clinically indicated; or metastatic castration resistant prostate cancer in patients whose disease has progressed on or after a docetaxel-based chemotherapy regimen. Further provided herein are methods further comprising administering a glucocorticoid. Further provided herein are methods further comprising administering prednisone, methyl prednisone, prednisolone or dexamethasone. Further provided herein are methods further comprising administering a chemotherapeutic agent.

Provided herein are methods for treating prostate cancer, comprising administering to a subject in need thereof a pharmaceutical composition, comprising (i) about 50 mg to about 250 mg of abiraterone acetate; (ii) at least one long chain fatty acid or at least one fatty acid glyceride; (iii) a surfactant; and (iv) optionally an antioxidant, optionally, wherein a total daily dose of abiraterone acetate administered to the subject is less than about 1000 mg. In some embodiments, the total daily dose of abiraterone acetate administered to the subject is less than about 500 mg. In some embodiments, the total daily dose of abiraterone acetate administered to the subject is less than about 250 mg.

Provided herein are methods for treating prostate cancer, comprising administering to a subject in need thereof a pharmaceutical composition, comprising about 50 mg to about 250 mg of abiraterone acetate; at least one long chain fatty acid or at least one fatty acid glyceride; a phospholipid; and optionally an antioxidant; wherein the total daily dose of abiraterone acetate administered to the subject is less than about 1000 mg. Further provided herein are methods wherein the total daily dose of abiraterone acetate administered to the subject is less than about 500 mg. Further provided herein are methods the total daily dose of abiraterone acetate administered to the subject is less than about 250 mg.

Provided herein are methods for preparing a pharmaceutical composition, comprising the steps: (a) combining an active pharmaceutical ingredient, a phospholipid, and, optionally, an antioxidant; (b) dissolving the mixture of step (a) in a long chain fatty acid or fatty acid glyceride; (c) enclosing the mixture of step (b) within a capsule. In some embodiments, the active pharmaceutical ingredient is selected from Table 1 or Table 2. Further provided herein are methods wherein the active pharmaceutical ingredient is abiraterone or pharmaceutically acceptable salt thereof or a pharmaceutically acceptable ester thereof. Further provided herein are methods wherein the pharmaceutically acceptable ester of the abiraterone is abiraterone acetate. Further provided herein are methods wherein the phospholipid is a component of lecithin. Further provided herein are methods wherein the long chain fatty acid is oleic acid. Further provided herein are compositions further comprising an antioxidant. Further provided herein are methods wherein the antioxidant is butylated hydroxyanisole or butylated hydroxytoluene or a combination thereof. Further provided herein are methods wherein the antioxidant comprises less than 1% of a total weight of the composition.

In one aspect, described herein is a pharmaceutical composition, comprising: (i) an active pharmaceutical ingredient or a pharmaceutically acceptable salt or ester thereof, wherein the active pharmaceutical ingredient has a calculated log P in octanol-water equal or greater than 4.0; (ii) at least one long chain fatty acid or at least one fatty acid glyceride; and (iii) a phospholipid. In some embodiments, the active pharmaceutical ingredient has a calculated log P equal or greater than 4.5. In some embodiments, the active pharmaceutical ingredient is selected from Table 1 or Table 2. In some embodiments, the active pharmaceutical ingredient has a calculated log P equal or greater than 5. In some embodiments, the solubility of the active pharmaceutical ingredient in the at least one long chain fatty acid or at least one fatty acid glyceride is at least 25 mg/mL. In some embodiments, the phospholipid is a phosphatidylcholine, a phosphatidylethanolamine, a phosphatidylinositol, a phosphatidylserine, a plasmalogen, a sphingomyelins, lecithin or phosphatidic acid. In some embodiments, the phospholipid is a component of lecithin. In some embodiments, the lecithin is isolated from egg yolk.

In one aspect, described herein is a pharmaceutical composition, comprising: (i) an active pharmaceutical ingredient having a weak base function group or a pharmaceutically acceptable salt thereof, wherein the active pharmaceutical ingredient has a calculated log P in octanol-water equal or greater than 4.0, and a pKa equal or greater than 3.0; (ii) at least one long chain fatty acid; and (iii) a phospholipid. In some embodiments, the active pharmaceutical ingredient is selected from Table 1 or Table 2. In some embodiments, the at least one long chain fatty acid is oleic acid. In some embodiments, the phospholipid is phosphatidylcholine. In some embodiments, the phospholipid is present in an amount of about 100 mg to about 200 mg. In some embodiments, the phospholipid comprises 10%-20% of the total weight of the composition. In some embodiments, the phospholipid comprises 14%-17% of the total weight of the composition. In some embodiments, a ratio by weight of the at least one long chain fatty acid to phospholipid is greater than 1.5. In some embodiments, the phospholipid comprises greater than 80% phosphatidylcholine.

In one aspect, described herein is a pharmaceutical composition, comprising: (i) abiraterone or a pharmaceutically acceptable salt or ester thereof; (ii) at least one long chain fatty acid or at least one fatty acid glyceride; and (iii) lecithin. In some embodiments, the pharmaceutically acceptable ester of the abiraterone is abiraterone acetate. In one aspect, described herein is a pharmaceutical composition, comprising: (i) a compound of the formula (I),

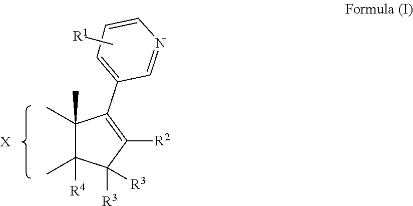

wherein X represents the residue of the A, B and C rings of a steroid selected from the group consisting of
androstan-3α- or 3β-ol,
androst-5-en-3α- or 3β-ol,
androst-4-en-3-one,
androst-2-ene,
androst-4-ene,
androst-5-ene,
androsta-5,7-dien-3α or 3β-ol,
androsta-1,4-dien-3-one,
estra-1,3,5[10]-trien-3-ol,
α-androstan-3-one,
androsta-3,5-diene,
androsta-3,5-diene-3-ol,
estra-1,3,5[10]-triene,
estra-1,3,5[10]-trien-3-ol,
5α-androstan-3-one,
androst-4-ene-3,11-dione,
6-fluoroandrost-4-ene-3-one, and
androstane-4-ene-3,6-dione,

each of which, where structurally permissible, can be further derivatized in one or more of the following ways:
to form 3-esters
to have one or more carbon or carbon ring double bonds in any of the 5,6-, 6,7-, 7,8-, 9,11- and 11,12-positions
as 3-oximes
as 3-methylenes
as 3-carboxylates
as 3-nitriles
as 3-nitros
as 3-desoxy derivatives
to have one or more hydroxy, halo, C₁₋₄-alkyl, trifluoro-methyl, C₁₋₄-alkoxy, C₁₋₄-alkanoyloxy, benzoyloxy, oxo, methylene or alkenyl substituents in the A, B, or C-ring,
to be 19-nor;
R¹ represents a hydrogen atom or an alkyl group of 1-4 carbon atoms;
R⁴ represents a hydrogen atom, a halogen atom or an alkyl group of 1 to 4 carbon atoms;
each of the R³ substituents independently represents a hydrogen atom or an alkyl or alkoxy group of 1-4 carbon atoms, a hydroxy group or an alkylcarbonyloxy group of 2 to 5 carbon atoms or together represent an oxo or methylene group, or R⁴ and one of the R³ groups together represent a double bond and the other R³ group represents a hydrogen atom or an alkyl group of 1 to 4 carbon atoms; and
R² represents a hydrogen atom, halogen atom, or an alkyl group of 1 to 4 carbon atoms, in the form of the free bases or pharmaceutically acceptable acid addition salts;

(ii) at least one long chain fatty acid or at least one fatty acid glyceride; and (iii) lecithin. In some embodiments, the compound is saturated and unsubstituted at the 11- and 12-positions. In some embodiments, the compound is selected from: 17-(3-pyridyl)androsta-5,16-dien-3β-ol, 17-(3-pyridyl)androsta-3,5,16-triene, 17-(3-pyridyl)androsta-4,16-dien-3-one, 17-(3-pyridyl)estra-1,3,5[10],16-tetraen-3-ol, 17-(3-pyridyl)-5α-androst-16-en-3α-ol, 17-(3-pyridyl)-5α-androst-16-en-3-one, 17-(3-pyridyl)-androsta-4,16-diene-3,11-dione, 17-(3-pyridyl)-androsta-3,5,16-trien-3-ol, 6α- and 6β-fluoro-17-(3-pyridyl)androsta-4,16-dien-3-one, 17-(3-pyridyl)androsta-4,16-dien-3,6-dione, 3α-trifluoromethyl-17-(3-pyridyl)androst-16-en-3β-ol, and their acid addition salts and 3-esters. In some embodiments, R¹ is hydrogen. In some embodiments, the compound is a compound of Table 2. In some embodiments, the compound is abiraterone acetate. In some embodiments, the abiraterone acetate is present in an amount of from 25 mg to 200 mg. In some embodiments, the abiraterone acetate is present in an amount of about 50 mg, about 100 mg or about 150 mg. In some embodiments, the at least one long chain fatty acid or the at least one fatty acid glyceride comprises greater than 11 carbon atoms. In some embodiments, the at least one long chain fatty acid or the at least one fatty acid glyceride consists of 12, 14, 16, 18, 20, 22 or 24 carbon atoms. In some embodiments, the at least one long chain fatty acid glyceride comprises one type of fatty acid glyceride. In some embodiments, the at least one long chain fatty acid glyceride comprises at least two different fatty acid glycerides. In some embodiments, the at least one long chain fatty acid comprises one type of long chain fatty acid. In some embodiments, the at least one long chain fatty acid comprises at least two different long chain fatty acids. In some embodiments, the at least one long chain fatty acid is unsaturated. In some embodiments, the at least one long chain fatty acid is saturated. In some embodiments, the at least one long chain fatty acid is Lauric acid, Myristic acid, Palmitic acid, Stearic acid, Arachidic acid, Behenic acid, Lignoceric acid or Cerotic acid. In some embodiments, the at least one long chain fatty acid is Myristoleic acid, Palmitoleic acid, Sapienic acid, Oleic acid, Elaidic acid, Vaccenic acid, Linoleic acid, Linolelaidic acid, γ-Linolenic acid, α-Linolenic acid, Stearidonic acid, Paullinic acid, Gondoic acid, Dihomo-γ-linolenic acid, Mead acid, Arachidonic acid, Eicosapentaenoic acid, Erucic acid, Docosatetraenoic acid, Docosahexaenoic acid or Nervonic acid. In some embodiments, the at least one long chain fatty acid or the at least one fatty acid glyceride is present in an amount of 100 mg to about 1000 mg. In some embodiments, the at least one long chain fatty acid or the at least one fatty acid glyceride is present in an amount of about 700 mg. In some embodiments, the at least one long chain fatty acid or the at least one fatty acid glyceride comprises 30%-95% of the total weight of the composition. In some embodiments, the at least one long chain fatty acid or the at least one fatty acid glyceride comprises 60%-80% of the total weight of the composition. In some embodiments, a ratio of the at least one long chain fatty acid or at least one fatty acid glyceride to the lecithin is greater than 1.5. In some embodiments, the lecithin is present in an amount of from 100 mg to 200 mg. In some embodiments, the at least one long chain fatty acid or at least one fatty acid glyceride is oleic acid. In some embodiments, the oleic acid is present in an amount of from 600 mg to 800 mg. In some embodiments, the abiraterone acetate, the at least one long chain fatty acid or at least one fatty acid glyceride, and the lecithin are present in a ratio by weight of 1:(4.66 to 14):(1 to 3), wherein the at least one long chain fatty acid or at least one fatty acid glyceride is oleic acid. In some embodiments, the abiraterone acetate, the at least one long chain fatty acid or at least one fatty acid glyceride, and the lecithin are present in a ratio by weight of about 1:14:3, about 1:7:1.5, or about 1:4.66:1, wherein the at least one long chain fatty acid or at least one fatty acid glyceride is oleic acid.

In some embodiments, a pharmaceutical composition described herein further comprises an antioxidant. In some embodiments, the antioxidant is vitamin E, butylated hydroxytoluene, butylated hydroxyanisole, ascorbyl palmitate, terbuteryl hydroquinone or a combination thereof. In some embodiments, the antioxidant is butylated hydroxyanisole or butylated hydroxytoluene or a combination thereof. In some embodiments, the antioxidant is present in an amount less than 1.5% of the total weight of the composition. In some embodiments, a pharmaceutical composition described herein is formulated for oral administration. In some embodiments, a pharmaceutical composition described herein is formulated for once daily dosing. In some embodiments, a pharmaceutical composition described herein is formulated for twice daily dosing.

In one aspect, described herein is a method for modulating androgen receptor activity, comprising administering to a subject in need of modulating androgen receptor activity, a pharmaceutical composition described herein. In one aspect, described herein is a method for treating prostate cancer, breast cancer, ovarian cancer, endometrial cancer, bladder cancer, pancreatic cancer, hepatocellular cancer, kidney cancer, liver cancer, salivary gland carcinoma, hair loss, acne, hirsutism, ovarian cysts, polycystic ovary disease, precocious puberty, spinal and bulbar muscular atrophy, or age-related macular degeneration, comprising administering to a subject in need thereof a pharmaceutical composition described herein. In one aspect, described herein is a method for treating prostate cancer, comprising administering to a subject in need thereof a pharmaceutical composition described herein. In some embodiments, the prostate cancer is selected from one or more of the following: castration-resistant prostate cancer; metastatic castration-resistant prostate cancer; castration-recurrent prostate cancer high-risk castration-sensitive prostate cancer; metastatic high-risk castration-sensitive prostate cancer; hormone-resistant prostate cancer; hormone-refractory prostate cancer; androgen-independent prostate cancer; androgen deprivation resistant prostate cancer; androgen ablation resistant prostate cancer; androgen depletion-independent prostate cancer; anti-androgen-recurrent prostate cancer; metastatic castration-resistant prostate cancer in patients who have already received prior chemotherapy containing docetaxel; newly diagnosed high risk metastatic hormone sensitive prostate cancer (mHSPC); metastatic castration resistant prostate cancer in patients who are asymptomatic or mildly symptomatic after failure of androgen deprivation therapy in whom chemotherapy is not yet clinically indicated; and metastatic castration resistant prostate cancer in patients whose disease has progressed on or after a docetaxel-based chemotherapy regimen. In some embodiments, the method comprises administering a glucocorticoid. In some embodiments, the method comprises administering prednisone, methyl prednisone, prednisolone or dexamethasone. In some embodiments, the method comprises administering a chemotherapeutic agent.

In one aspect, described herein is a method for treating prostate cancer, comprising administering to a subject in need thereof a pharmaceutical composition, comprising (i) about 50 mg to about 250 mg of abiraterone acetate; (ii) at least one long chain fatty acid or at least one fatty acid glyceride; (iii) a surfactant; and (iv) optionally an antioxidant, optionally, wherein a total daily dose of abiraterone acetate administered to the subject is less than about 1000 mg. In some embodiments, the total daily dose of abiraterone acetate administered to the subject is less than about 500 mg. In some embodiments, the total daily dose of abiraterone acetate administered to the subject is less than about 250 mg.

Provided herein are uses of the described pharmaceutical composition in preparation of a medicament for modulating androgen receptor activity or treating prostate cancer. Provided herein are kits comprising the described pharmaceutical composition. Provided herein are pharmaceutical compositions as described herein formulated into a form of capsule.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 plot of in vivo oral bioavailability (as AUC) (X-axis) versus amount of abiraterone in the aqueous phase as a percentile (Y-axis).

### DETAILED DESCRIPTION

The present invention is generally directed to compositions comprising pharmaceutically active agents that are useful as therapeutics that alleviate, abate or eliminate one or more conditions in a subject in need thereof, as further described herein. In particular, described herein are pharmaceutical compositions, their preparation method and use, where the pharmaceutical compositions comprise a lipophilic active pharmaceutical ingredient (API), a long chain fatty acid or long chain fatty acid glyceride, and a phospholipid in a combination such that the API has improved bioavailability compared to the API alone.

### Definitions

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The singular forms “a”, “an”, and “the” include plural referents unless the context clearly dictates otherwise. Thus, for example, reference to “the surfactant” includes reference to one or more specific surfactants, reference to “an antioxidant” includes reference to one or more of such additives.

The term “subject” as used herein refers to a mammal (e.g., a human, mouse, rat, guinea pig, dog, cat, horse, cow, pig, or non-human primate, such as a monkey, chimpanzee or baboon).

“AUC” as used herein refers to the area under the plasma drug concentration-versus-time curve extrapolated from zero time to infinity. “Cₘₐₓ” as used herein refers to the highest drug concentration observed in plasma following an extravascular dose of drug. “Tₘₐₓ” as used herein refers to the time after administration of a drug when the maximum plasma concentration is reached.

“Effective amount,” and “sufficient amount” may be used interchangeably, and refer to an amount of a substance that is sufficient to achieve an intended purpose or objective.

The term “steroid” herein includes any compound having the steroidal B and C rings, but in which all or part of the A ring is missing e.g. ring not closed (lacking the 2- or 3-position C-atom or both) or takes the form of a cyclopentane or cyclohexane ring. It also includes azasteroids having a ring nitrogen atom in place of a ring carbon atom, especially in the A-ring such as in 4-azasteroids.

A “therapeutically effective amount” when used in connection with a pharmaceutical composition described herein is an amount of one or more pharmaceutically active agent(s) sufficient to produce a therapeutic result in a subject in need thereof. An “amount” of one or more components in the pharmaceutical composition refers to an amount per unit dose.

“Therapeutically equivalent” when used in connection with a pharmaceutical composition described herein refers to an amount or quantity of a pharmaceutically acceptable salt or ester of a pharmaceutically active agent that is equivalent to the therapeutically effective amount of the free base or alcohol of the pharmaceutically active agent.

### In Vitro and In Vivo Assessment of Lipid Based Formulations

A recent review discussed progress in developing in vitro in vivo correlations for lipid-based formulations (see Feeney et al., “50 years of oral lipid-based formulations: Provenance, progress and future perspectives”, Advanced Drug Delivery Reviews, 2016, 101, 167-194). Section 6.2 of this article, titled “Does in vitro LBF digestion data correlate with in vivo absorption?” states: “Subjecting the formulation to an in vitro digestion challenge test has therefore become more common and allows some estimation of the GI disposition of drug after the lipid carrier has been hydrolyzed”. It continues, “Consistent with the rationale for formulation dispersion tests . . . maximizing aqueous phase drug concentrations (and minimizing drug precipitation) is expected to maximize the likelihood of robust drug absorption from lip-based formulations in vivo. . . . Digestion testing therefore provides a useful screening tool to identify formulations with a strong chance of good absorption in vivo—but may eliminate some formulation that might otherwise provide for useful absorption”. Given this knowledge, those of skill in the arts related to lipid formulations, might be motivated to perform this assay and then preferentially select those formulations that maximize aqueous phase drug concentrations, as it remains unclear whether those formulations with poor aqueous phase drug concentrations will necessarily have poor in vivo absorption. Described herein are formulations with low aqueous phase drug concentrations, yet good in vivo absorption qualities. In some embodiments, provided herein are abiraterone acetate formulations that demonstrated low aqueous phase drug concentrations in the digestion assay, yet presented good in vivo absorption qualities.

### Active Pharmaceutical Ingredient (API)

Various embodiments described herein are directed to compositions comprising an effective amount of an active pharmaceutical agent (API). “Active pharmaceutical agent”, “API”, “drug”, “pharmaceutically active agent”, “bioactive agent”, “therapeutic agent”, and “active agent” and the like may be used interchangeably and refer to a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates, hydrates, radioisotopes, etc.

The partition-coefficient (P) as referenced herein is a ratio of concentrations of a compound between two immiscible solvent phases at equilibrium. Most commonly, one of the solvents is water and the other is hydrophobic, typically 1-octanol. The logarithm of the ratio is log P, as shown below (conventionally the lipophilic phase is the numerator and hydrophilic phase is the denominator):

$$log {P}_{octanol / water} = log ( \frac{{[ solute ]}_{o c t a n o l}}{{[ solute ]}_{w a t e r}} )$$

log P is a measure of lipophilicity or hydrophobicity. Hydrophobicity affects drug absorption, bioavailability, hydrophobic drug-receptor interactions, metabolism of molecules, and toxicity. Hydrophilic compounds are soluble in water (“water-loving”) and polar solvents. Lipophilic compounds are less soluble in water (“water-fearing” or hydrophobic) and polar solvents, but are more soluble in organic solvents. Thus: Low hydrophilicity=high lipophilicity=high log P=poor absorption. High hydrophilicity=low lipophilicity=low log P=good absorption.
Those of skill in the art are familiar with Lapinsky's rule of five, which suggests for compounds to have a reasonable likelihood of being well absorbed their log P must be less than 5.0.

Partition coefficients can be measured experimentally or estimated via calculation. Various methods for calculating (or predicting) log P have been developed, typically by fitting calculated log P values with experimentally measured log P values for training sets of thousands of molecules, mostly drug-like. log P calculations are considered very robust and accurately process many organic molecules. For example, over 50% of molecules log P is predicted with error of less than 0.25, while over 80% with error of less than 0.5. Less than 3.5% of structures are predicted with an error greater 1.0. To distinguish from a measured log P, a calculated log P is sometimes written as clog P.

In some embodiments, an API described herein is lipophilic. In some embodiments, the API is insoluble in polar solvents. In some embodiments, the API is insoluble in aqueous media. In some embodiments, the API is insoluble in water.

In some embodiments, an API described herein has a calculated log P of at least 2.5. In some embodiments, the API has a calculated log P of at least 2.6. In some embodiments, the API has a calculated log P of at least 2.7. In some embodiments, the API has a calculated log P of at least 2.8. In some embodiments, the API has a calculated log P of at least 2.9. In some embodiments, the API has a calculated log P of greater than 3.0. In some embodiments, the API has a calculated log P of greater than 3.1. In some embodiments, the API has a calculated log P of greater than 3.2. In some embodiments, the API has a calculated log P of greater than 3.3. In some embodiments, the API has a calculated log P of greater than 3.4. In some embodiments, the API has a calculated log P of greater than 3.5. In some embodiments, the API has a calculated log P of greater than 3.6. In some embodiments, the API has a calculated log P of greater than 3.7. In some embodiments, the API has a calculated log P of greater than 3.8. In some embodiments, the API has a calculated log P of greater than 3.9. In some embodiments, the API has a calculated log P of greater than 4.0. In some embodiments, the API has a calculated log P of greater than 4.1. In some embodiments, the API has a calculated log P of greater than 4.2. In some embodiments, the API has a calculated log P of greater than 4.3. In some embodiments, the API has a calculated log P of greater than 4.4. In some embodiments, the API has a calculated log P of greater than 4.5. In some embodiments, the API has a calculated log P of greater than 4.6. In some embodiments, the API has a calculated log P of greater than 4.7. In some embodiments, the API has a calculated log P of greater than 4.8. In some embodiments, the API has a calculated log P of greater than 4.9. In some embodiments, the API has a calculated log P of greater than 5.0. In some embodiments, the API has a calculated log P of greater than 5.1. In some embodiments, the API has a calculated log P of greater than 5.2. In some embodiments, the API has a calculated log P of greater than 5.3. In some embodiments, the API has a calculated log P of greater than 5.4. In some embodiments, the API has a calculated log P of greater than 5.5. In some embodiments, the API has a calculated log P of greater than 5.6. In some embodiments, the API has a calculated log P of greater than 5.7. In some embodiments, the API has a calculated log P of greater than 5.8. In some embodiments, the API has a calculated log P of greater than 5.9. In some embodiments, the API has a calculated log P of greater than 6.0. In some embodiments, the API has a calculated log P of greater than 6.1. In some embodiments, the API has a calculated log P of greater than 6.2. In some embodiments, the API has a calculated log P of greater than 6.3. In some embodiments, the API has a calculated log P of greater than 6.4. In some embodiments, the API has a calculated log P of greater than 6.5. In some embodiments, the API has a calculated log P of greater than 6.6. In some embodiments, the API has a calculated log P of greater than 6.7. In some embodiments, the API has a calculated log P of greater than 6.8. In some embodiments, the API has a calculated log P of greater than 6.9. In some embodiments, the API has a calculated log P of greater than 7.0. Exemplary small molecule APIs include, without limitation, those listed in Table 1.

TABLE 1

| Name | clogP |
|---|---|
| Abiraterone Acetate | 5.12 (LogP) |
| Nilotinib | 4.4 |
| Chlordiazepoxide | 3.79 |
| Naringenin | 2.44 |
| Clofibric acid | 2.82 |
| Hesperetin | 2.29 |
| Cinchonidine | 2.49 |
| Quinine | 2.99 |
| Tramadol | 3.10 |
| Lormetazepam | 2.40 |
| Trazodone | 3.85 |
| Diltiazem | 3.65 |
| Brompheniramine | 3.30 |
| Alprenolol | 2.65 |
| Propranolol | 2.75 |
| Diazepam | 2.96 |
| Apigenin | 2.91 |
| Venlafaxine | 3.27 |
| Rosiglitazone | 3.02 |
| Pyrilamine | 3.23 |
| Quetiapine | 2.99 |
| Ketoprofen | 2.76 |
| Chlorphenamine | 3.15 |
| Fenbufen | 3.14 |
| Naproxen | 2.82 |
| Warfarin | 2.90 |
| Diphenhydramine | 3.45 |
| Bupivacaine | 3.69 |
| Clotiazepam | 3.03 |
| Chrysin | 3.56 |
| Valsartan | 3.63 |
| Haloperidol | 3.85 |
| Flurbiprofen | 3.75 |
| Progesterone | 3.78 |
| Celecoxib | 4.37 |
| Flurazepam | 4.22 |
| Glimepiride | 3.96 |
| Ibuprofen | 3.68 |
| Atorvastatin | 4.46 |
| Indometacin | 4.18 |
| Nortriptyline | 4.32 |
| Loratadine | 5.05 |
| Fluoxetine | 4.57 |
| Diclofenac | 4.73 |
| Clopidogrel | 4.21 |
| Duloxetine | 4.26 |
| Penbutolol | 3.64 |
| Maprotiline | 4.52 |
| Imipramine | 5.04 |
| Amitriptyline | 4.85 |
| Cyproheptadine | 5.30 |
| Sertraline | 5.35 |
| Flufenamic acid | 5.53 |
| Chlorpromazine | 5.30 |
| Miconazole | 5.81 |
| Rimonabant | 6.47 |
| Clofazimine | 7.70 |
| Enzalutamide | 4.51 |

An acid dissociation constant, Ka, (or acidity constant) is a measure of the strength of an acid or base in solution, typically water. It is the equilibrium constant for the chemical dissociation of acids and bases. In aqueous solution, the equilibrium of acid dissociation is written:

HA+H₂O [symbol] A⁻+H₃O⁺

where HA is an acid that dissociates into A⁻ (the conjugate base of the acid) and a hydrogen ion (which combines with a water molecule to make a hydronium ion, H₃O). The dissociation constant can also be written with the H₂O removed:

$$HA ⇋ {A}^{-} + {H}^{+}$$

$${K}_{a} = \frac{[ {A}^{-} ] [ {H}^{+} ]}{[ HA ]}$$

The equilibrium of the dissociation of the conjugate acid of a base is written:

BH⁺+H₂O [symbol] B+H₃O⁺

where BH⁺ (the conjugate acid of the base) dissociates into B (the free base), and a hydrogen ion, which combines with a water molecule to form a hydronium ion, H₃O⁺. The dissociation constant can also be written with the H₂O removed:

$${BH}^{+} ⇋ B + {H}^{+}$$

$${K}_{b} = \frac{[ B ] [ {H}^{+} ]}{[ {BH}^{+} ]}$$

pKₐ, the logarithmic value of Kₐ, is more often used to express the strength/weakness of acids or the conjugate acid of bases:

pKₐ=−log₁₀(Kₐ)

The more positive the value of pKa, the smaller the extent of dissociation, and the weaker the acid. In general, for acids:

pKa=−2 to 12→weak acid (little or only partial dissociation in water)
pKa<−2→strong acid (completely or mostly dissociated in water)
while for bases:
pKₐ<12→weak base (little or only partial dissociation in water)
pKa>12→strong base (completely or mostly dissociated in water)

In some embodiments, an API described herein is a weak base. In some embodiments, an API described herein comprises a weak base functional group. In some embodiments, an API described herein has a pKa of equal or greater than 3.0. In some embodiments, the API has a pKa of equal or greater than 3.5. In some embodiments, the API has a pKa of equal or greater than 4.0. In some embodiments, the API has a pKa of equal or greater than 4.5. In some embodiments, the API has a pKa of equal or greater than 5.0.

In some embodiments, an API described herein is present in the form of a free base. In some embodiments, an API described herein is present in the form of a pharmaceutically acceptable salt. As used herein, a pharmaceutically acceptable salt includes, but is not limited to, metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. In some embodiments the API is abiraterone or a pharmaceutically acceptable salt or ester thereof. In some embodiments, the API is abiraterone acetate. In some embodiments, the API is a pharmaceutically acceptable salt of abiraterone acetate. In some embodiments, the API is one listed in Tables 1 or 2, or a pharmaceutically acceptable salt thereof.

Pharmaceutically acceptable salts include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentafluoropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically acceptable salt. In some embodiments the API is abiraterone or a pharmaceutically acceptable salt or ester thereof. In some embodiments, the pharmaceutically acceptable ester of abiraterone is abiraterone acetate. In some embodiments, the API is one listed in Tables 1 or 2, or a pharmaceutically acceptable salt or ester thereof.

In some embodiments, an API described herein is present in a herein-described pharmaceutical composition in an amount of at least 10 mg. In some embodiments, the API is present in an amount of at least 20 mg. In some embodiments, the API is present in an amount of at least 25 mg. In some embodiments, the API is present in an amount of at least 30 mg. In some embodiments, the API is present in an amount of at least 40 mg. In some embodiments, the API is present in an amount of at least 50 mg. In some embodiments, the API is present in an amount of at least 60 mg. In some embodiments, the API is present in an amount of at least 70 mg. In some embodiments, the API is present in an amount of at least 75 mg. In some embodiments, the API is present in an amount of at least 80 mg. In some embodiments, the API is present in an amount of at least 90 mg. In some embodiments, the API is present in an amount of at least 100 mg. In some embodiments, the API is present in an amount of at least 110 mg. In some embodiments, the API is present in an amount of at least 120 mg. In some embodiments, the API is present in an amount of at least 125 mg. In some embodiments, the API is present in an amount of at least 130 mg. In some embodiments, the API is present in an amount of at least 140 mg. In some embodiments, the API is present in an amount of at least 150 mg. In some embodiments, the API is present in an amount of at least 160 mg. In some embodiments, the API is present in an amount of at least 170 mg. In some embodiments, the API is present in an amount of at least 175 mg. In some embodiments, the API is present in an amount of at least 180 mg. In some embodiments, the API is present in an amount of at least 190 mg. In some embodiments, the API is present in an amount of at least 200 mg. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof. In some embodiments, the API is clofazimine and nilotinib or palbociclib or a pharmaceutically acceptable salt thereof.

In some embodiments, an API described herein is present in a herein-described pharmaceutical composition in an amount of about 10 mg. In some embodiments, the API is present in an amount of about 20 mg. In some embodiments, the API is present in an amount of about 25 mg. In some embodiments, the API is present in an amount of about 30 mg. In some embodiments, the API is present in an amount of about 40 mg. In some embodiments, the API is present in an amount of about 50 mg. In some embodiments, the API is present in an amount of about 60 mg. In some embodiments, the API is present in an amount of about 70 mg. In some embodiments, the API is present in an amount of about 75 mg. In some embodiments, the API is present in an amount of about 80 mg. In some embodiments, the API is present in an amount of about 90 mg. In some embodiments, the API is present in an amount of about 100 mg. In some embodiments, the API is present in an amount of about 110 mg. In some embodiments, the API is present in an amount of about 120 mg. In some embodiments, the API is present in an amount of about 125 mg. In some embodiments, the API is present in an amount of about 130 mg. In some embodiments, the API is present in an amount of about 140 mg. In some embodiments, the API is present in an amount of about 150 mg. In some embodiments, the API is present in an amount of about 160 mg. In some embodiments, the API is present in an amount of about 170 mg. In some embodiments, the API is present in an amount of about 175 mg. In some embodiments, the API is present in an amount of about 180 mg. In some embodiments, the API is present in an amount of about 190 mg. In some embodiments, the API is present in an amount of about 200 mg. In some embodiments, the API is present in an amount of about 225 mg. In some embodiments, the API is present in an amount of about 250 mg. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, an API described herein is present in a herein-described pharmaceutical composition in an amount of no more than 1000 mg. In some embodiments, the API is present in an amount of no more than 750 mg. In some embodiments, the API is present in an amount of no more than 500 mg. In some embodiments, the API is present in an amount of no more than 400 mg. In some embodiments, the API is present in an amount of no more than 300 mg. In some embodiments, the API is present in an amount of no more than 250 mg. In some embodiments, the API is present in an amount of no more than 225 mg. In some embodiments, the API is present in an amount of no more than 200 mg. In some embodiments, the API is present in an amount of no more than 175 mg. In some embodiments, the API is present in an amount of no more than 150 mg. In some embodiments, the API is present in an amount of no more than 125 mg. In some embodiments, the API is present in an amount of no more than 100 mg. In some embodiments, the API is present in an amount of no more than 90 mg. In some embodiments, the API is present in an amount of no more than 80 mg. In some embodiments, the API is present in an amount of no more than 75 mg. In some embodiments, the API is present in an amount of no more than 60 mg. In some embodiments, the API is present in an amount of no more than 55 mg. In some embodiments, the API is present in an amount of no more than 50 mg. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, an API described herein is present in a herein-described pharmaceutical composition in an amount of 10 mg to 1000 mg. In some embodiments, the API is present in an amount of 20 mg to 500 mg. In some embodiments, the API is present in an amount of 20 mg to 400 mg. In some embodiments, the API is present in an amount of 20 mg to 300 mg. In some embodiments, the API is present in an amount of 25 mg to 250 mg. In some embodiments, the API is present in an amount of 30 mg to 200 mg. In some embodiments, the API is present in an amount of about 50 mg, about 100 mg or about 150 mg. In some embodiments, the API is present in an amount of 50 mg, 100 mg or 150 mg. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, an API described herein comprises about 5% of the total weight of the pharmaceutical composition. In some embodiments, the API comprises about 10% of the total weight of the composition. In some embodiments, the API comprises about 15% of the total weight of the composition. In some embodiments, the API comprises about 20% of the total weight of the composition. In some embodiments, the API comprises about 25% of the total weight of the composition. In some embodiments, the API comprises about 30% of the total weight of the composition. In some embodiments, the API comprises about 40% of the total weight of the composition. In some embodiments, the API comprises about 50% of the total weight of the composition. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, an API described herein comprises up to 5% of the total weight of the pharmaceutical composition. In some embodiments, the API comprises up to 10% of the total weight of the composition. In some embodiments, the API comprises up to 15% of the total weight of the composition. In some embodiments, the API comprises up to 20% of the total weight of the composition. In some embodiments, the API comprises up to 50% of the total weight of the composition. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, an API described herein comprises about 5.5% of the total weight of the pharmaceutical composition. In some embodiments, the API comprises about 10.5% of the total weight of the composition. In some embodiments, the API comprises about 15% of the total weight of the composition. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, the solubility of an API described herein in long chain fatty acids or derivatives thereof is at least 25 mg/mL. In some embodiments, the solubility of the API in long chain fatty acids or derivatives thereof is at least 30 mg/mL. In some embodiments, the solubility of the API in long chain fatty acids or derivatives thereof is at least 40 mg/mL. In some embodiments, the solubility of the API in long chain fatty acids or derivatives thereof is at least 50 mg/mL. In some embodiments, the solubility of the API in long chain fatty acids or derivatives thereof is at least 75 mg/mL. In some embodiments, the solubility of the API in long chain fatty acids or derivatives thereof is at least 100 mg/mL. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In one aspect, an API described herein is a compound of the formula (I),

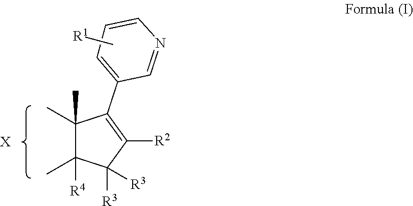

wherein X represents the residue of the A, B and C rings of a steroid selected from the group consisting of
androstan-3α- or 3β-ol,
androst-5-en-3α- or 3β-ol,
androst-4-en-3-one,
androst-2-ene,
androst-4-ene,
androst-5-ene,
androsta-5,7-dien-3α or 3β-ol,
androsta-1,4-dien-3-one,
estra-1,3,5[10]-trien-3-ol,
α-androstan-3-one,
androsta-3,5-diene,
androsta-3,5-diene-3-ol,
estra-1,3,5[10]-triene,
estra-1,3,5[10]-trien-3-ol,
5α-androstan-3-one,
androst-4-ene-3,11-dione,
6-fluoroandrost-4-ene-3-one, and
androstane-4-ene-3,6-dione,

each of which, where structurally permissible, can be further derivatized in one or more of the following ways:
to form 3-esters
to have one or more carbon or carbon ring double bonds in any of the 5,6-, 6,7-, 7,8-, 9,11- and 11,12-positions
as 3-oximes
as 3-methylenes
as 3-carboxylates
as 3-nitriles
as 3-nitros
as 3-desoxy derivatives
to have one or more hydroxy, halo, C₁₋₄-alkyl, trifluoro-methyl, C₁₋₄-alkoxy, C₁₋₄-alkanoyloxy, benzoyloxy, oxo, methylene or alkenyl substituents in the A, B, or C-ring,
to be 19-nor;
R¹ represents a hydrogen atom or an alkyl group of 1-4 carbon atoms;
R⁴ represents a hydrogen atom, a halogen atom or an alkyl group of 1 to 4 carbon atoms; each of the R³ substituents independently represents a hydrogen atom or an alkyl or alkoxy group of 1-4 carbon atoms, a hydroxy group or an alkylcarbonyloxy group of 2 to 5 carbon atoms or together represent an oxo or methylene group, or R⁴ and one of the R³ groups together represent a double bond and the other R³ group represents a hydrogen atom or an alkyl group of 1 to 4 carbon atoms; and
R² represents a hydrogen atom, halogen atom, or an alkyl group of 1 to 4 carbon atoms, in the form of the free bases or pharmaceutically acceptable acid addition salts.

In some embodiments of a compound of Formula (I), the compound is saturated and unsubstituted at the 11- and 12-positions. In some embodiments of a compound of Formula (I), the compound is saturated at the 11- and 12-positions. In some embodiments of a compound of Formula (I), the compound is unsubstituted at the 11- and 12-positions. In some embodiments of a compound of Formula (I), the compound is substituted at the 11- and/or the 12-position. In some embodiments, the compound comprises 11-oxo or 11β-hydroxy. In some embodiments of a compound of Formula (I), R¹ is hydrogen. In some embodiments of a compound of Formula (I), R¹ is methyl. In some embodiments of a compound of Formula (I), R² is hydrogen, alkyl of 1 to 3 carbon atoms, fluorine, chlorine, bromine, or iodine. In some embodiments of a compound of Formula (I), R² is hydrogen. In some embodiments of a compound of Formula (I), each of RS is independently hydrogen or alkyl of 1 to 3 carbon atoms. In some embodiments of a compound of Formula (I), each of R⁵ is hydrogen. In some embodiments of a compound of Formula (I), R⁴ is hydrogen or an alkyl group of 1 to 4 carbon atoms.

In some embodiments of a compound of Formula (I), the compound is selected from: 17-(3-pyridyl)androsta-5,16-dien-3β-ol, 17-(3-pyridyl)androsta-3,5,16-triene, 17-(3-pyridyl)androsta-4,16-dien-3-one, 17-(3-pyridyl)estra-1,3,5[10], 16-tetraen-3-ol, 17-(3-pyridyl)-5α-androst-16-en-3α-ol, 17-(3-pyridyl)-5α-androst-16-en-3-one, 17-(3-pyridyl)-androsta-4,16-diene-3,11-dione, 17-(3-pyridyl)-androsta-3,5,16-trien-3-ol, 6α- and 6β-fluoro-17-(3-pyridyl)androsta-4,16-dien-3-one, 17-(3-pyridyl)androsta-4,16-dien-3,6-dione, 3α-trifluoromethyl-17-(3-pyridyl)androst-16-en-3β-ol, and their acid addition salts and 3-esters. In some embodiments of a compound of Formula (I), the compound is 30-alkanoyloxy-17-(3-pyridyl)androsta-5,16-dienes in which the alkanoyloxy group has from 2 to 4 carbon atoms. In some embodiments of a compound of Formula (I), the compound is abiraterone acetate.

In some embodiments of a compound of Formula (I), the compound has a structure of Formula (II),

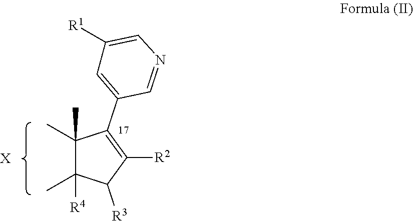

wherein X, R¹, R², R³ and R⁴ are described in Formula (I).

In some embodiments of a compound of Formula (II), the compound is selected from Table 2.

TABLE 2
Exemplary Compounds Having a Structure
of Formula (II) and 3-esters thereof.*

| X in Formula (II) represents the | | | | |
|---|---|---|---|---|
| residue of the A, B and C rings of a | | | | |
| steroid selected from | R¹ | R² | R³ | R⁴ |
| androstan-3α-ol | H | H | H | H |
| androstan-3β-ol | H | H | H | H |
| androst-5-en-3α-ol | H | H | H | H |
| androst-5-en-3β-ol | H | H | H | H |
| androst-4-en-3-one | H | H | H | H |
| androst-2-ene | H | H | H | H |
| androst-4-ene | H | H | H | H |
| androst-5-ene, | H | H | H | H |
| androsta-5,7-dien-3α-ol | H | H | H | H |
| androsta-5,7-dien-3β-ol | H | H | H | H |
| androsta-1,4-dien-3-one | H | H | H | H |
| estra-1,3,5[10]-trien-3-ol | H | H | H | H |
| α-androstan-3-one | H | H | H | H |
| androsta-3,5-diene | H | H | H | H |
| androsta-3,5-diene-3-ol | H | H | H | H |
| estra-1,3,5[10]-triene | H | H | H | H |
| estra-1,3,5[10]-trien-3-ol | H | H | H | H |
| 5α-androstan-3-one | H | H | H | H |
| androst-4-ene-3,11-dione | H | H | H | H |
| 6-fluoroandrost-4-ene-3-one | H | H | H | H |
| androstane-4-ene-3,6-dione | H | H | H | H |
| androstan-3α-ol | methyl | H | H | H |
| androstan-3β-ol | methyl | H | H | H |
| androst-5-en-3α-ol | methyl | H | H | H |
| androst-5-en-3β-ol | methyl | H | H | H |
| androst-4-en-3-one | methyl | H | H | H |
| androst-2-ene | methyl | H | H | H |
| androst-4-ene | methyl | H | H | H |
| androst-5-ene, | methyl | H | H | H |
| androsta-5,7-dien-3α-ol | methyl | H | H | H |
| androsta-5,7-dien-3β-ol | methyl | H | H | H |
| androsta-1,4-dien-3-one | methyl | H | H | H |
| estra-1,3,5[10]-trien-3-ol | methyl | H | H | H |
| α-androstan-3-one | methyl | H | H | H |
| androsta-3,5-diene | methyl | H | H | H |
| androsta-3,5-diene-3-ol | methyl | H | H | H |
| estra-1,3,5[10]-triene | methyl | H | H | H |
| estra-1,3,5[10]-trien-3-ol | methyl | H | H | H |
| 5α-androstan-3-one | methyl | H | H | H |
| androst-4-ene-3,11-dione | methyl | H | H | H |
| 6-fluoroandrost-4-ene-3-one | methyl | H | H | H |
| androstane-4-ene-3,6-dione | methyl | H | H | H |

*Each of the compounds of Table 2, where structurally permissible, can be further derivatized to form 3-esters, all of which are encompassed by Table 2.

In some embodiments of a compound of Formula (I), the compound has a structure of Formula (III),

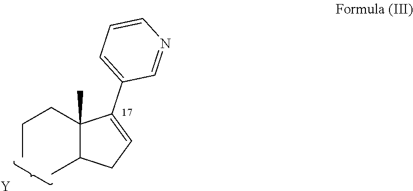

wherein Y is

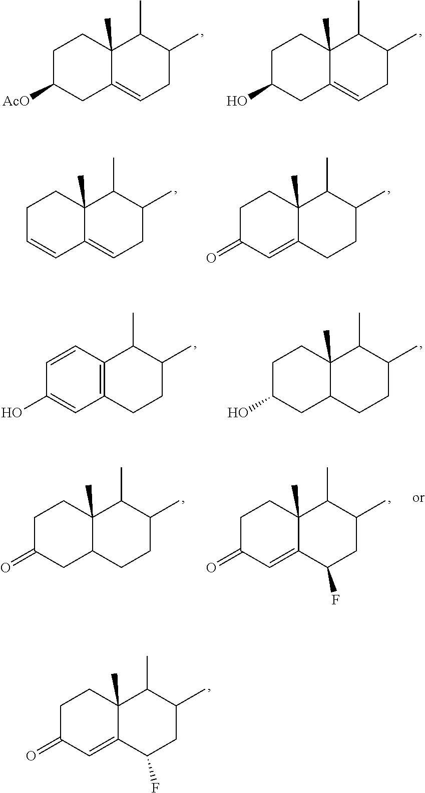

each of which, where structurally permissible, can be further derivatized to form 3-esters. In one aspect, provided herein is a pharmaceutical composition comprising an API, wherein the API is a compound having a structure of Formula (I), Formula (II), or Formula (III) as described above.

### Long Chain Fatty Acids and Fatty Acid Glycerides

In some embodiments, pharmaceutical compositions described herein comprise a lipophilic API, a long chain fatty acid or a long chain fatty acid glyceride, a phospholipid, and optionally, an antioxidant. In some embodiments, the API has a clog P greater than 3.5 or greater than 4, a solvent with solubility of API with a clog P greater than 4 and an oral absorption enhancer. In some embodiments, the solvent is a fatty acid, a fatty acid ester, a vegetable oil, a polyethylene glycol fatty acid ester, ethanol and diethylene glycol ether, or combination thereof. In some embodiments, the solvent is a fatty acid, a medium chain fatty acid, glycerol, castor oil, ethanol and diethylene glycol ether or combination thereof. In some embodiments, the solvent is a fatty acid, a medium chain fatty acid glyceride, or combination thereof. In some embodiments, the pharmaceutical compositions comprise an API with a clog P greater than 4, and a fatty acid. Fatty acids are carboxylic acids consisting of a hydrocarbon chain and a terminal carboxy (—COOH) group. The hydrocarbon chain may be saturated (containing no carbon-carbon double bonds) or unsaturated, including monounsaturated (containing one carbon-carbon double bond) or polyunsaturated (containing more than one carbon-carbon double bond). Fatty acids can contain carbon chains that are straight (or unbranched), carbon chains, or they can contain branched carbon chains. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof. In some embodiments, long chain fatty acids are carboxylic acids comprising at least 12 carbon atoms. In some embodiments, the long chain fatty acid comprises at least 11 carbon atoms. In some embodiments, the long chain fatty acid comprises at least 12 carbon atoms. In some embodiments, the long chain fatty acid comprises at least 13 carbon atoms. In some embodiments, the long chain fatty acid comprises at least 14 carbon atoms. In some embodiments, the long chain fatty acid comprises at least 15 carbon atoms. In some embodiments, the long chain fatty acid comprises at least 16 carbon atoms. In some embodiments, the long chain fatty acid comprises at least 17 carbon atoms. In some embodiments, the long chain fatty acid comprises at least 18 carbon atoms. In some embodiments, the long chain fatty acid comprises at least 19 carbon atoms. In some embodiments, the long chain fatty acid comprises at least 20 carbon atoms. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof. In some embodiments, the long chain fatty acid comprises at least 16 carbon atoms. In some embodiments, the long chain fatty acid comprises 16 carbon atoms. In some embodiments, the long chain fatty acid contains 16 carbon atoms. Examples of straight chain, saturated fatty acids include, but are not limited to those listed in Table 3.

TABLE 3

| Common name | Chemical structure |
|---|---|
| Lauric acid | CH₃(CH₂)₁₀COOH |
| Myristic acid | CH₃(CH₂)₁₂COOH |
| Palmitic acid | CH₃(CH₂)₁₄COOH |
| Stearic acid | CH₃(CH₂)₁₆COOH |
| Arachidic acid | CH₃(CH₂)₁₈COOH |
| Behenic acid | CH₃(CH₂)₂₀COOH |
| Lignoceric acid | CH₃(CH₂)₂₂COOH |
| Cerotic acid | CH₃(CH₂)₂₄COOH |

Examples of straight chain, unsaturated fatty acids include, but are not limited to, those listed in Table 4.

TABLE 4

| Name | #C | Structural Formula |
|---|---|---|
| Myristoleic acid | 14 | CH₃(CH2)₃CH═CH(CH₂)₇COOH |
| Palmitoleic acid | 16 | CH₃(CH₂)₅CH═CH(CH₂)₇COOH |
| Sapienic acid | 16 | CH₃(CH₂)₈CH═CH(CH₂)₄COOH |
| Oleic acid | 18 | CH₃(CH₂)₇CH═CH(CH₂)₇COOH |
| Elaidic acid | 18 | CH₃(CH₂)₇CH═CH(CH₂)₇COOH |
| Vaccenic acid | 18 | CH₃(CH₂)₅CH═CH(CH₂)₉COOH |
| Linoleic acid | 18 | CH₃(CH₂)₄CH═CHCH₂CH═CH(CH₂)₇COOH |
| Linolelaidic acid | 18 | CH₃(CH₂)₄CH═CHCH₂CH═CH(CH₂)₇COOH |
| γ-Linolenic acid | 18 | CH₃(CH₂)₄CH═CHCH₂CH═CHCH₂CH═CH(CH₂)₄COOH |
| α-Linolenic acid | 18 | CH₃CH₂CH═CHCH₂CH═CHCH₂CH═CH(CH₂)₇COOH |
| Stearidonic acid | 18 | CH₃CH₂CH═CHCH₂CH═CHCH₂CH═CHCH₂CH═CH(CH₂)₄COOH |
| Paullinic acid | 20 | CH₃(CH₂)₅CH═CH(CH₂)₁₁COOH |
| Gondoic acid | 20 | CH₃(CH₂)₇CH═CH(CH₂)₉COOH |
| Dihomo-γ-linolenic acid | 20 | CH₃(CH₂)₄CH═CHCH₂CH═CHCH₂CH═CH(CH₂)₆COOH |
| Mead acid | 20 | CH₃(CH₂)₇CH═CHCH₂CH═CHCH₂CH═CH(CH₂)₃COOH |
| Arachidonic acid | 20 | CH₃(CH₂)₄CH═CHCH₂CH═CHCH₂CH═CHCH₂CH═CH(CH₂)₃COOH |
| Eicosapentaenoic acid | 20 | CH₃CH₂CH═CHCH₂CH═CHCH₂CH═CHCH₂CH═CHCH₂CH═CH(CH₂)₃COOH |
| Erucic acid | 22 | CH₃(CH₂)₇CH═CH(CH₂)₁₁COOH |
| Docosatetraenoic acid | 22 | CH₃(CH₂)₄CH═CHCH₂CH═CHCH₂CH═CHCH₂CH═CH(CH₂)₅COOH |
| Docosahexaenoic acid | 22 | CH₃CH₂CH═CHCH₂CH═CHCH₂CH═CHCH₂CH═CHCH₂CH═CHCH₂CH═CH(CH₂)₂COOH |
| Nervonic acid | 24 | CH₃(CH₂)₇CH═CH(CH₂)₁₃COOH |

In some embodiments, pharmaceutical compositions described herein include a lipophilic API, a long chain fatty acid or a long chain fatty acid glyceride, a surfactant and, optionally, and antioxidant. In some embodiments, the surfactant is a phospholipid. In some embodiments, the glycerides are fatty acid esters of glycerol.

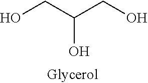

Glycerides may be monoglycerides, diglycerides and triglycerides, wherein glycerol is esterified with one, two, or three fatty acids. Other terms in the art for triglycerides include TG's, triacylglycerols, TAG's, triacylglycerides, fats and the like and are used interchangeably herein. Glycerides may be further modified by substitution onto one or more for the hydrocarbon chains.

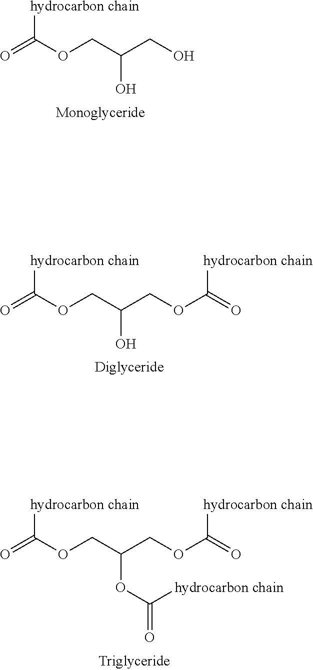

Examples of mono- and diglycerides include, but are not limited to Monopalmitolein, Monoelaidin, Monocaproin, Monocaprylin, Monocaprin, Monolaurin, Glyceryl monomyristate, Glyceryl monooleate, Glyceryl monooleate, Glycerol monooleate/linoleate, Glycerol monolinoleate, Glyceryl ricinoleate, Glyceryl monolaurate, Glycerol monopalmitate, Glycerol monostearate, Glyceryl mono-, dioleate, Glyceryl palmitic/stearic, Glyceryl acetate, Glyceryl laurate, Glyceryl citrate/lactate/oleate/linoleate, Glyceryl caprylate, Glyceryl caprylate/caprate, Caprylic acid mono, diglycerides, Caprylic/capric glycerides, Mono- and diacetylated monoglycerides, Glyceryl monostearate, Lactic acid esters of mono, diglycerides, Dicaproin, Dicaprin, Dioctanoin, Dimyristin, Dipalmitin, Distearin, Glyceryl dilaurate, Glyceryl dioleate, Glycerol esters of fatty acids, 1,2 and 1,3-diolein, Dielaidin, Dilinolein, and combinations thereof. Other fatty acids include stearyl alcohol, capric acid, caprylic acid, lauric acid, myristic acid, stearic acid, oleic acid, linoleic acid, linolenic acid, arachnidoic acid, behenic acid, and their corresponding pharmaceutically acceptable salts. Preferred fatty acid and fatty alcohol derivatives include sodium dioctyl sulfosuccinate, sodium lauryl sulfate, amide esters (e.g., lauric acid diethanolamide, sodium lauryl sarcosinate, lauroyl carnitine, palmitoyl carnitine and myristoyl carnitine), esters with hydroxy-acids (e.g., sodium stearoyl lactylate); sugar esters [e.g., lauryl lactate, glucose monocaprylate, diglucose monocaprylate, sucrose laurate, sorbitan monolaurate (Arlacel® 20), sorbitanmonopalmitate (Span-40), sorbitan monooleate (Span-80), sorbitan monostearate and sorbitantristearate, lower alcohol fatty acid esters [e.g., ethyl oleate (Crodamol EO), isopropyl myristate (Crodamol IPM) and isopropyl palmitate (Crodamol IPP)], esters with propylene glycol [e.g., propylene glycol monolaurate (Lauroglycol FCC), propylene glycol ricinoleate (Propymuls), propylene glycol monooleate (Myverol® P-06), propylene glycol monocaprylate (Capryol® 90), propylene glycol dicaprylate/dicaprate (Captex® 200) and propylene glycol dioctanoate (Captex 800)], esters with glycerol [e.g., glyceryl monooleate (Peceol), glyceryl ricinoleate, glyceryl laurate, glyceryl dilaurate (Capmul® GDL), glyceryl dioleate (Capmul GDO), glycerol monolinoleate (Maisine®), glyceryl mono/dioleate (Capmul GMO-K), glyceryl caprylate/caprate (Capmul MCM), caprylic acid mono/diglycerides (Imwitor® 988), mono- and diacetylated monoglycerides (Myvacet® 9-45)], triglycerides [e.g., corn oil, almond oil, soybean oil, coconut oil, castor oil, hydrogenated castor oil, hydrogenated coconut oil, Pureco 100, Hydrokote AP5, Captex 300, 350, Miglyol 812, Miglyol 818 and Gelucire 33/01)], mixtures of propylene glycol esters and glycerol esters [e.g., mixture of oleic acid esters of propylene glycol and glycerol (Arlacel 186)], and polyglycerized fatty acids such as polyglyceryl oleate (Plurol® Oleique), polyglyceryl-2 dioleate (Nikko] DGDO), polyglyceryl-10 trioleate, polyglyceryl-10 laurate (Nikkol Decaglyn 1-L), polyglyceryl-10 oleate (Nikkol Decaglyn 1-O), and polyglyceryl-10 mono, dioleate (Caprol® PEG 860).

Additional fatty acid derivatives include polyethoxylated fatty acids, (e.g., PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate), PEG-fatty acid diesters (e.g., PEG-20 dilaurate, PEG-20 dioleate, PEG-20 distearate, PEG-32 dilaurate and PEG-32 dioleate), PEG-fatty acid mono- and di-ester mixtures, polyethylene glycol glycerol fatty acid esters (e.g., PEGylated glycerol 12 acyloxy-stearate, PEG-20 glyceryl laurate, PEG-30 glyceryl laurate, PEG-40 glyceryl laurate, PEG-20 glyceryl oleate and PEG-30 glyceryl oleate) and alcohol-oil transesterification products [e.g., polyoxyl 40 castor oil (Cremophor® RH40), polyoxyl 35 castor oil (Cremophor EL or Incrocas 35), PEG-25 trioleate (TAGAT® TO), PEG-60 corn glycerides (Crovol M70), PEG-60 almond oil (Crovol A70), PEG 40 palm kernel oil (Crovol PK70), PEG-50 castor oil (Emalex C-50), PEG-50 hydrogenated castor oil (Emalex HC-50), PEG-60 hydrogenated castor oil (Cremophor RH60), PEG-8 caprylic/capric glycerides (Labrasol®), lauroyl macrogol 32 glycerides (Gelucire® 44/14), linoleoyl macrogoglycerides (Labrafil®), stearoyl macrogol-32 glycerides (Gelucire 50/13), and PEG-6 caprylic/capric glycerides (Softigen® 767)].

In some embodiments, the long chain fatty acid described herein comprises bile acid and sterol derivatives. Exemplary bile acid and sterol derivatives include, but are not limited to, cholate, ursodeoxycholate, chenodeoxycholate, taurochenodeoxycholate, tauroursodeoxycholate, glycochenodeoxycholate, glycoursodeoxycholate, sterols and sterol esters or ethers such as PEG-24 cholesterol ether.

In some embodiments, the long chain fatty acid or long chain fatty acid glyceride comprises a tocol derivative. Tocol derivatives include derivatives of substances with the tocol structure [2 methyl-2-(4,8,12-trimethyltridecyl)chroman-6-ol] or the tocotrienol structure [2 methyl-2-(4,8,12-trimethyltrideca-3,7,11-trienyl)chroman-6-of]. In particular, the mono-, di-, trimethyl-tocols, commonly known as tocopherols and their organic acid esters such as the acetate, nicotinate, succinate, and polyethylnene glycol succinate esters are included. For example, a-tocopherol acetate, a-tocopherol nicotinate, a tocopherol succinate, a.-tocopherol polyethyleneglycol (200-8000 MW) succinate, a tocopherol polyethylene glycol 400 succinate, dl-a-tocopherol polyethyleneglycol 1000 succinate, and d-a-tocopherol polyethyleneglycol 1000 succinate (Vitamin E TPGS, Eastman Chemical Co.) are included. For the practice of this invention the mixed racemic forms (e.g. all racemic or dl-) as well as the pure enantiomers (e.g. d-, l- or RRR-) are suitable. Preferred tocol derivative include a-tocopherol esters and a polyethoxylated a-tocopherol esters. More specific preferred tocol derivatives include a-tocopherol, a-tocopherol acetate, a-tocopherol nicotinoate, a-tocopherol succinate, a-tocopherol polyethyleneglycol succinate, a-tocopherol polyethyleneglycol (200-8000 MW) succinate, a-tocopherol polyethylene glycol 400 succinate, a-tocopherol polyethyleneglycol 1000 succinate, dl-a-tocopherol polyethyleneglycol 1000 succinate, or d-a-tocopherol polyethyleneglycol 1000 succinate.

As used herein, the term “long chain lipid solvent” refers to pharmaceutically acceptable lipid solvents comprising twelve or more carbon atoms. In some embodiments, the long chain lipid solvents are able to dissolve the therapeutic amount of API. Examples of long chain lipid solvents include, but are not limited to fatty acids, glycerides, vegetable oils, hydro-vegetable oils, animal oils, PEGylated glycerides, Vitamin E derivatives and combinations thereof. In some embodiments, the long chain lipid solvent comprises at least twelve carbon atoms.

### Surfactant

In some embodiments, pharmaceutical compositions described herein comprise a lipophilic API, a long chain fatty acid or a long chain fatty acid glyceride, a surfactant and, optionally, and antioxidant. In some embodiments, the surfactants are compounds or mixture of compounds comprising a hydrophobic group (usually a hydrocarbon chain) and a hydrophilic group. They may perform one or more roles including solubility enhancer, bioavailability enhancer, stability enhancer, antioxidant and emulsifying agent. Other terms in the art for surfactants include emulsifier, emulsifying agent, surface-active agent, wetting agent, suspending agent and the like. Examples of surfactants include, but are not limited to, phospholipids, sucrose esters of fatty acids, polyoxyl stearate, polyoxyethylene hydrogenated castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, sodium dodecyl sulfate, lauromacrogol Arlasolve, Poloxamers, Labrafil, Labrasol, Tween 80 and the like. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

Lipids can refer to fatty acids and derivatives thereof that are soluble in non-polar, or organic, solvents while insoluble in water and polar solvents. Categories of lipids include, but are not limited to, fatty acids, phospholipids, sphingolipids, saccharolipids, polyketides, sterol lipids, prenol lipids and the like. In some embodiments, phospholipids are made up of glycerol to which is attached a phosphate group and two fatty acids. Other terms in the art for phospholipids include glycerophospholipids, phosphoglycerides, diacylglycerides and the like. The phosphate group can be unmodified (i.e. in the structure below R═H) or modified by attachment (i.e. in the structure below R≠H) to simple organic molecules such as, but not limited to choline, ethanolamine or serine.

Phospholipids may be further modified by substitution onto one or more for the hydrocarbon chains.

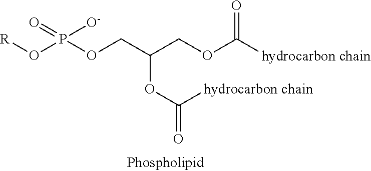

In some embodiments, phospholipids are selected from glycerophospholipid, sphingolipid, and/or phospholipid derivatives. In some embodiments, glycerophospholipids include, but are not limited to phosphatidylcholine, phosphatidyl ethanolamine, phosphatidyl ethanolamine, phosphatidyl serine, phosphatidyl glycerol, diphosphatidylglycerol, phosphatidylinositol, and mixtures thereof. Phospholipid derivatives according to the present invention include, but are not limited to dioleoylphosphatidylcholine, dimyristoylphosphatidylcholine, dipentadeanoylphosphatidylcholine, dilauroylphosphatidylchoine, dipalmitoylphosphatidylcholine (DPPC), distearoylphosphatidylcholine (DSPC), diarachidonyiphosphatidylcholine (DAPC), dioleoylphosphatidylethanolamine, dipalmitoylphosphatidylethanolamine (DPPE), and distearoylphosphatidylethanolamine (DSPE), disteraoylphosphatidylglycerol (DSPG), phosphatidylinositol, dipalmitoylphosphatidic acid (DPPA), distearoylphosphatidic acid (DSPA), and the like, and mixtures thereof.

In some embodiments, phosphatidylcholines are phospholipids wherein a choline group (Me₃N⁺—CH₂—CH₂—O—) is attached to the phosphate group.

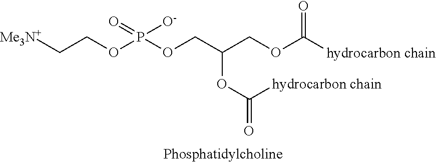

A non-limiting example of a phosphatidylcholine is 1-oleoyl-2-palmitoyl-phosphatidyl choline, as shown below:

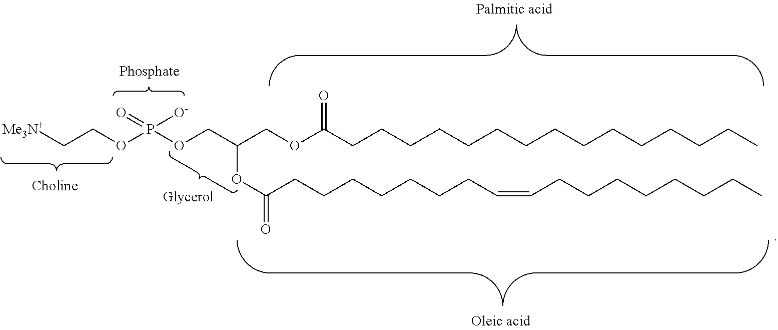

The USP 40 definition of lecithin is “a complex mixture of acetone-insoluble phosphatides, which consist chiefly of phophatidylcholine, phosphatidylethanolamine, phosphatilinositol, and phosphatidic acid, present in conjunction with various amounts of other substances such as triglycerides, fatty acids, and carbohydrates, as separated from the crude vegetable oil source”. In some embodiments, lecithin is a mixture of phospholipids. Lecithins can be isolated from various sources including, but not limited to eggs, soybeans, milk, marine sources, rapeseed, cottonseed and sunflower.

In some embodiments, the surfactant is a phospholipid. In some embodiments, the phospholipid is phosphatidylcholine. In some embodiments, the phospholipid is a mixture comprising phosphatidylcholine. In some embodiments, the surfactant is lecithin. In some embodiments, the phosphatidylcholine is lecithin. In some embodiments, the lecithin contains more than 25% of phosphatidylcholine. In some embodiments, the lecithin contains more than 80% of phosphatidylcholine. In some embodiments, the phosphatidylcholine is from egg origin. In some embodiments, the phosphatidylcholine is from or soybean origin.

In some embodiments, the phospholipid is present in a herein-described pharmaceutical composition in an amount of no less than 5 mg. In some embodiments, the phospholipid is present in an amount of no less than 10 mg. In some embodiments, the phospholipid is present in an amount of no less than 15 mg. In some embodiments, the phospholipid is present in an amount of no less than 20 mg. In some embodiments, the surfactant is present in an amount of no less than 25 mg. In some embodiments, the phospholipid is present in an amount of no less than 30 mg. In some embodiments, the phospholipid is present in an amount of no less than 40 mg. In some embodiments, the phospholipid is present in an amount of no less than 50 mg. In some embodiments, the phospholipid is present in an amount of no less than 60 mg. In some embodiments, the phospholipid is present in an amount of no less than 70 mg. In some embodiments, the phospholipid is present in an amount of no less than 75 mg. In some embodiments, the phospholipid is present in an amount of no less than 80 mg. In some embodiments, the phospholipid is present in an amount of no less than 90 mg. In some embodiments, the phospholipid is present in an amount of no less than 100 mg. In some embodiments, the phospholipid is present in an amount of no less than 110 mg. In some embodiments, the phospholipid is present in an amount of no less than 120 mg. In some embodiments, the phospholipid is present in an amount of no less than 125 mg. In some embodiments, the phospholipid is present in an amount of no less than 130 mg. In some embodiments, the phospholipid is present in an amount of no less than 140 mg. In some embodiments, the phospholipid is present in an amount of no less than 150 mg. In some embodiments, the phospholipid is present in an amount of no less than 160 mg. In some embodiments, the phospholipid is present in an amount of no less than 170 mg. In some embodiments, the phospholipid is present in an amount of no less than 175 mg. In some embodiments, the phospholipid is present in an amount of no less than 180 mg. In some embodiments, the phospholipid is present in an amount of no less than 190 mg. In some embodiments, the phospholipid is present in an amount of no less than 200 mg. In some embodiments, the phospholipid is present in an amount of no less than 225 mg. In some embodiments, the phospholipid is present in an amount of no less than 250 mg. In some embodiments, pharmaceutical compositions described herein include a lipophilic API, a long chain fatty acid or a long chain fatty acid glyceride, a phospholipid, and optionally, an antioxidant. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, the phospholipid is present in a herein-described pharmaceutical composition in an amount of about 5 mg. In some embodiments, the phospholipid is present in an amount of about 10 mg. In some embodiments, the phospholipid is present in an amount of about 15 mg. In some embodiments, the phospholipid is present in an amount of about 20 mg. In some embodiments, the phospholipid is present in an amount of about 25 mg. In some embodiments, the phospholipid is present in an amount of about 30 mg. In some embodiments, the phospholipid is present in an amount of about 40 mg. In some embodiments, the phospholipid is present in an amount of about 50 mg. In some embodiments, the phospholipid is present in an amount of about 60 mg. In some embodiments, the phospholipid is present in an amount of about 70 mg. In some embodiments, the phospholipid is present in an amount of about 75 mg. In some embodiments, the phospholipid is present in an amount of about 80 mg. In some embodiments, the phospholipid is present in an amount of about 90 mg. In some embodiments, the phospholipid is present in an amount of about 100 mg. In some embodiments, the phospholipid is present in an amount of about 110 mg. In some embodiments, the phospholipid is present in an amount of about 120 mg. In some embodiments, the phospholipid is present in an amount of about 125 mg. In some embodiments, the phospholipid is present in an amount of about 130 mg. In some embodiments, the phospholipid is present in an amount of about 140 mg. In some embodiments, the phospholipid is present in an amount of about 150 mg. In some embodiments, the phospholipid is present in an amount of about 160 mg. In some embodiments, the phospholipid is present in an amount of about 170 mg. In some embodiments, the phospholipid is present in an amount of about 175 mg. In some embodiments, the phospholipid is present in an amount of about 180 mg. In some embodiments, the phospholipid is present in an amount of about 190 mg. In some embodiments, the phospholipid is present in an amount of about 200 mg. In some embodiments, the phospholipid is present in an amount of about 225 mg. In some embodiments, the phospholipid is present in an amount of about 250 mg. In some embodiments, pharmaceutical compositions described herein include a lipophilic API, a long chain fatty acid or a long chain fatty acid glyceride, a phospholipid, and optionally, an antioxidant. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, the phospholipid is present in a herein-described pharmaceutical composition in an amount of no more than 25 mg. In some embodiments, the phospholipid is present in an amount of no more than 30 mg. In some embodiments, the phospholipid is present in an amount of no more than 40 mg. In some embodiments, the phospholipid is present in an amount of no more than 50 mg. In some embodiments, the phospholipid is present in an amount of no more than 60 mg. In some embodiments, the phospholipid is present in an amount of no more than 70 mg. In some embodiments, the phospholipid is present in an amount of no more than 75 mg. In some embodiments, the phospholipid is present in an amount of no more than 80 mg. In some embodiments, the phospholipid is present in an amount of no more than 90 mg. In some embodiments, the phospholipid is present in an amount of no more than 100 mg. In some embodiments, the phospholipid is present in an amount of no more than 110 mg. In some embodiments, the phospholipid is present in an amount of no more than 120 mg. In some embodiments, the phospholipid is present in an amount of no more than 125 mg. In some embodiments, the phospholipid is present in an amount of no more than 130 mg. In some embodiments, the phospholipid is present in an amount of no more than 140 mg. In some embodiments, the phospholipid is present in an amount of no more than 150 mg. In some embodiments, the phospholipid is present in an amount of no more than 160 mg. In some embodiments, the phospholipid is present in an amount of no more than 170 mg. In some embodiments, the phospholipid is present in an amount of no more than 175 mg. In some embodiments, the phospholipid is present in an amount of no more than 180 mg. In some embodiments, the phospholipid is present in an amount of no more than 190 mg. In some embodiments, the phospholipid is present in an amount of no more than 200 mg. In some embodiments, the phospholipid is present in an amount of no more than 225 mg. In some embodiments, the phospholipid is present in an amount of no more than 250 mg. In some embodiments, the phospholipid is present in an amount of no more than 300 mg. In some embodiments, the phospholipid is present in an amount of no more than 500 mg. In some embodiments, pharmaceutical compositions described herein include a lipophilic API, a long chain fatty acid or a long chain fatty acid glyceride, a phospholipid, and optionally, an antioxidant. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, the phospholipid is present in a herein-described pharmaceutical composition in an amount of 50 mg to 500 mg. In some embodiments, the phospholipid is present in an amount of 75 mg to 300 mg. In some embodiments, the phospholipid is present in an amount of 100 mg to 200 mg. In some embodiments, the phospholipid is present in an amount of 125 mg to 175 mg. In some embodiments, the phospholipid is present in an amount of about 50, 75, 100, 125, 150, 175, 200, 225, 250, 300, 350, 400, 450, or 500 mg. In some embodiments, the phospholipid comprises 5%-50% of the total weight of the pharmaceutical composition. In some embodiments, the phospholipid comprises 10%-20% of the total weight of the composition. In some embodiments, the phospholipid comprises 14%-17% of the total weight of the composition. In some embodiments, the phospholipid comprises about 15% of the total weight of the composition. In some embodiments, the phospholipid comprises about 16% of the total weight of the composition. In some embodiments, the phospholipid comprises about 17% of the total weight of the composition. In some embodiments, the ratio by weight of the fatty acid or fatty acid glyceride to lecithin is greater than 0.75. In some embodiments, the ratio by weight of the fatty acid or fatty acid glyceride to lecithin is greater than 1.0. In some embodiments, the ratio by weight of the fatty acid or fatty acid glyceride to lecithin is greater than 1.1. In some embodiments, the ratio by weight of the fatty acid or fatty acid glyceride to lecithin is greater 1.2. In some embodiments, the ratio by weight of the fatty acid or fatty acid glyceride to lecithin is greater 1.3. In some embodiments, the ratio by weight of the fatty acid or fatty acid glyceride to lecithin is greater 1.4. In some embodiments, the ratio by weight of the fatty acid or fatty acid glyceride to lecithin is greater than 1.5. In some embodiments, pharmaceutical compositions described herein include a lipophilic API, a long chain fatty acid or a long chain fatty acid glyceride, a phospholipid, and optionally, an antioxidant. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

### Antioxidant

Compositions described herein may optionally comprise an antioxidant. In some embodiments, antioxidants are used to reduce the oxidation or degradation of APIs and excipients in the composition. In some embodiments, the antioxidant is selected from ascorbic acid, ascorbyl palmitate, butylated hydroxy anisole (BHA), butylated hydroxyl toluene (BHT), citric acid, cysteine, gallic acid, guaiac resin, methionine, phosphoric acid, potassium metabisulfite, propyl gallate, sesamol, sodium edate, sodium sulfite, sodium bisulfite, sodium metabisulfite, sodium thiosulfate, terbuteryl hydroquinone, tartaric acid, tertiary butyl hydroquinone, Vitamin E (tocopherol), thioglycerol, thioglycollic acid, or a combination thereof. In some embodiments, the antioxidant is vitamin E, butylated hydroxytoluene, butylated hydroxyanisole, or a combination thereof.

In some embodiments, the antioxidant is present in a herein-described pharmaceutical composition in an amount less than 2.0% of the total weight of the pharmaceutical composition. In some embodiments, the antioxidant is present in an amount less than 1.75% of the total weight of the composition. In some embodiments, the antioxidant is present in an amount less than 1.5% of the total weight of the composition. In some embodiments, the antioxidant is present in an amount less than 1.25% of the total weight of the composition. In some embodiments, the antioxidant is present in an amount less than 1.0% of the total weight of the composition. In some embodiments, the antioxidant comprises about 0.1% of the total weight of the composition. In some embodiments, the antioxidant comprises about 0.2% of the total weight of the composition. In some embodiments, pharmaceutical compositions described herein include a lipophilic API, a long chain fatty acid or a long chain fatty acid glyceride, a phospholipid, and optionally, an antioxidant. In some embodiments, the antioxidant comprises about 0.3% of the total weight of the composition. In some embodiments, the antioxidant comprises about 0.4% of the total weight of the composition. In some embodiments, the antioxidant comprises about 0.5% of the total weight of the composition. In some embodiments, the antioxidant comprises about 0.6% of the total weight of the composition. In some embodiments, the antioxidant comprises about 0.7% of the total weight of the composition. In some embodiments, the antioxidant comprises about 0.8% of the total weight of the composition. In some embodiments, the antioxidant comprises about 0.9% of the total weight of the composition. In some embodiments, the antioxidant comprises about 1.0% of the total weight of the composition. In some embodiments, the antioxidant comprises about 1.1% of the total weight of the composition. In some embodiments, the antioxidant comprises about 1.2% of the total weight of the composition. In some embodiments, the antioxidant comprises about 1.25% of the total weight of the composition. In some embodiments, the antioxidant is present in an amount of from about 0.01% to about 2% of the total weight of the composition. In some embodiments, the antioxidant is present in an amount of from about 0.1% to about 1.75% of the total weight of the composition. In some embodiments, the antioxidant is present in an amount of from about 0.2% to about 1.5% of the total weight of the composition. In some embodiments, the antioxidant is present in an amount of from about 0.3% to about 1.25% of the total weight of the composition. In some embodiments, the antioxidant is present in an amount of from about 0.4% to about 1.1% of the total weight of the composition. In some embodiments, pharmaceutical compositions described herein comprise a lipophilic API, a long chain fatty acid or a long chain fatty acid glyceride, a phospholipid, and optionally, an antioxidant. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

### Other Additives

In some embodiments, pharmaceutical compositions described herein comprise a lipophilic API, a long chain fatty acid or a long chain fatty acid glyceride, a phospholipid, and optionally, an antioxidant and/or another additive. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof. In some embodiments, other additives conventionally used in pharmaceutical compositions can be included, and these additives are well known in the art. Such additives include, but are not limited to, anti-adherents (anti-sticking agents, glidants, flow promoters, lubricants) (e.g., talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate) anticoagulants (e.g., acetylated monoglycerides), antifoaming agents (e.g., long-chain alcohols and silicone derivatives), antioxidants (e.g., BHT, BHA, gallic acid, propyl gallate, ascorbic acid, ascorbyl palmitate, 4hydroxymethyl-2,6-di-tert-butyl phenol, tocopherol, etc.), binders (adhesives), i.e., agents that impart cohesive properties to powdered materials through particle-particle bonding (e.g., matrix binders (dry starch, dry sugars), film binders (PVP, starch paste, celluloses, bentonite, sucrose)), chemical binders (e.g., polymeric cellulose derivatives, such as carboxy methyl cellulose, HPC, HPMC, etc., sugar syrups, corn syrup, water soluble polysaccharides (e.g., acacia, tragacanth, guar, alginates, etc), gelatin, gelatin hydrolysate, agar, sucrose, dextrose, non-cellulosic binders (e.g., PVP, PEG, vinyl pyrrolidone copolymers, pregelatinized starch, sorbitol, glucose, etc.), bufferants, where the acid is a pharmaceutically acceptable acid, (e.g., hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, etc.) and where the base is a pharmaceutically acceptable base (e.g., an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine), or a pharmaceutically acceptable salt of acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, parabromophenylsulfonic acid, propionic acid, p-toluenesulfonic acid, salicylic acid, stearic acid, succinic acid, tannic acid, tartaric acid, thioglycolic acid, toluenesulfonic acid, and uric acid, chelating agents (e.g., EDTA and EDTA salts), coagulants (e.g., alginates) colorants or opaquants (e.g., titanium dioxide, food dyes, lakes, natural vegetable colorants, iron oxides, silicates, sulfates, magnesium hydroxide and aluminum hydroxide), coolants (e.g. halogenated hydrocarbons (e.g., trichloroethane, trichloroethylene, dichloromethane, fluorotrichloromethane), diethylether and liquid nitrogen) cryoprotectants (e.g., trehelose, phosphates, citric acid, tartaric acid, gelatin, dextran, mannitol, etc.), diluents or fillers (e.g., lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose disintegrants or super disintegrants (e.g., croscarmellose sodium, starch, starch derivatives, clays, gums, cellulose, cellulose derivatives, alginates, crosslinked polyvinylpyrrolidone, sodium starch glycolate and microcrystalline cellulose), hydrogen bonding agents (e.g., magnesium oxide), flavorants or desensitizers (e.g., spray-dried flavors, essential oils and ethyl vanillin), ion-exchange resins (e.g., styrene/divinyl benzene copolymers, and quaternary ammonium compounds), plasticizers (e.g., polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate), preservatives (e.g., ascorbic acid, boric acid, sorbic acid, benzoic acid, and salts thereof, parabens, phenols, benzyl alcohol, and quaternary ammonium compounds), solvents (e.g., alcohols, ketones, esters, chlorinated hydrocarbons and water) sweeteners, including natural sweeteners (e.g., maltose, sucrose, glucose, sorbitol, glycerin and dextrins), and artificial sweeteners (e.g., aspartame, saccharine and saccharine salts) and thickeners (viscosity modifiers, thickening agents), (e.g., sugars, polyvinylpyrrolidone, cellulosics, polymers and alginates). Additives can also be materials such as proteins (e.g., collagen, gelatin, Zein, gluten, mussel protein, lipoprotein), carbohydrates (e.g., alginates, carrageenan, cellulose derivatives, pectin, starch, chitosan), gums (e.g., xanthan gum, gum arabic), spermaceti, natural or synthetic waxes, carnuaba wax, fatty acids (e.g., stearic acid, hydroxystearic acid), fatty alcohols, sugars, shellacs, such as those based on sugars (e.g., lactose, sucrose, dextrose) or starches, polysaccharide-based polymers (e.g., maltodextrin and maltodextrin derivatives, dextrates, cyclodextrin and cyclodextrin derivatives), cellulosic-based polymers (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate, trimellitate, carboxymethylethyl cellulose, hydroxypropylmethyl cellulose phthalate), inorganics, (e.g., dicalcium phosphate, hydroxyapitite, tricalcium phosphate, talc and titania), polyols (e.g., mannitol, xylitol and sorbitol polyethylene glycol esters) and polymers (e.g., alginates, poly(lactide coglycolide), gelatin, crosslinked gelatin and agar-agar).

In some embodiments, pharmaceutical compositions described herein include a lipophilic API, a long chain fatty acid or a long chain fatty acid glyceride, a phospholipid, and optionally, an adsorbent. Many adsorbents are solid, porous or super porous adsorption materials. They comprise numerous micro- or nano-pores within their structures, resulting in very large surface areas, for example, greater than 500 m²/g. Exemplary absorbents include, without limitation, silica, active carbon, magnesium aluminum silicate, and diatomite. In further embodiments, the pharmaceutical composition is in an oral dosage form, e.g., a tablet or capsule.

It should be appreciated that there is considerable overlap between the above listed components in common usage, since a given component is often classified differently by different practitioners in the field, or is commonly used for any of several different functions, or may have differing functions depending on the levels in the composition. Thus, the above-listed components should be taken as merely exemplary, and not limiting, of the types of components that can be included in compositions of the present invention.

### Oral Dosage Forms

The present pharmaceutical compositions can take the form of solutions, suspensions, emulsion, tablets, pills, pellets, capsules, capsules containing liquids, powders, suppositories, emulsions, suspensions, or any other form suitable for use. Preferred pharmaceutical compositions are formulated for oral delivery. In some embodiments, the pharmaceutically acceptable vehicle is a capsule. Capsules may be hard capsules or soft capsules, push-fit capsules made of gelatin, as well as soft, sealed capsules made of gelatin and a plasticizer (such as glycerol or sorbitol). In some embodiments, the capsule contains about 1000 mg of the pharmaceutical composition. In some embodiments, the capsule contains less than 1000 mg of the pharmaceutical composition. Capsules can be of any size. Examples of standard sizes include, but are not limited to those listed in Table 5 (#000, #00, #0, #1, #2, #3, #4, and #5). In some embodiments, the pharmaceutical composition is in the dosage form of a liquid filled into a hard capsule. In some embodiments, the pharmaceutical composition is in the dosage form of a liquid filled into a soft capsule. In some embodiments, the pharmaceutical composition comprises an adsorbent. In some embodiments, the pharmaceutical composition comprises an adsorbent in the dosage form of a tablet. In some embodiments, the pharmaceutical composition is in the dosage form of a multilayer tablet. In some embodiments, the tablet has one, two, three, four or more layers. In some embodiments, the tablet has an inner core and an outer core. See, e.g., Remington's Pharmaceutical Sciences, page 1658-1659 (Alfonso Gennaro ed., Mack Publishing Company, Easton Pa., 18th ed., 1990), which is incorporated by reference. In some embodiments, the capsules used herein are of size #00 or #0.

TABLE 5

| | Volume | Locked | External |
|---|---|---|---|
| Size | (mL) | length (mm) | diameter (mm) |
| 000 | 1.37 | 26.1 | 9.9 |
| 00 | 0.91 | 23.3 | 8.5 |
| 0 | 0.68 | 21.7 | 7.6 |
| 1 | 0.50 | 19.4 | 6.9 |
| 2 | 0.37 | 18.0 | 6.3 |
| 3 | 0.30 | 15.9 | 5.8 |
| 4 | 0.21 | 14.3 | 5.3 |

### Methods of Administration

Pharmaceutical compositions described herein may be administered for the treatment or prevention of diseases. When used to treat or prevent diseases or disorders, pharmaceutical compositions may be administered or applied singly, or in combination with other agents. Pharmaceutical compositions may also be administered or applied singly, in combination with other pharmaceutically active agents. Provided herein are methods of treatment and prophylaxis by administration to a subject in need of such treatment of a therapeutically effective amount of a pharmaceutical composition of the invention. The subject can be an animal, e.g., a mammal such as a human. In some embodiments, pharmaceutical compositions described herein include a lipophilic API, a long chain fatty acid or a long chain fatty acid glyceride, a phospholipid, and optionally, an antioxidant. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, the pharmaceutical compositions are administered orally. In some embodiments, the pharmaceutical compositions are administered in an oral liquid, semi-liquid or semisolid dosage form. In some embodiments, the pharmaceutical compositions are administered as a solid oral dosage form. In some embodiments, the pharmaceutical compositions are administered as a liquid oral dosage form. In some embodiments, the pharmaceutical compositions are administered as a pill, tablet, chewable tablet, specialty tablet, buccal tablet, sub-lingual tablet, orally-disintegrating tablet, capsule, gel capsule, soft gel capsule, hard gel capsule, specialty capsule, buccal capsule, sub-lingual capsule, orally-disintegrating capsule, powder, granule, crystal or orally dispersible film. In some embodiments, the pharmaceutical compositions are administered as a liquid or a capsule. In some embodiments, the pharmaceutical compositions are administered as a soft gel capsule. In some embodiments, the pharmaceutical compositions are administered as a hard gel capsule. In some embodiments, the pharmaceutical compositions are administered at regular intervals such as once daily, twice daily, or on a weekly basis.

### Conditions

In some embodiments, herein-described pharmaceutical compositions may be used to modulate androgen receptor activity in a subject in need of modulating androgen receptor activity. In some embodiments, pharmaceutical compositions described herein comprise a lipophilic API, a long chain fatty acid or a long chain fatty acid glyceride, a phospholipid, and optionally, an antioxidant. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, a pharmaceutical composition described herein may be used to treat or prevent cancer. In some embodiments, the pharmaceutical compositions may be used to treat or prevent prostate cancer, breast cancer, ovarian cancer, endometrial cancer, bladder cancer, pancreatic cancer, hepatocellular cancer, kidney cancer, liver cancer, salivary gland carcinoma, hair loss, acne, hirsutism, ovarian cysts, polycystic ovary disease, precocious puberty, spinal and bulbar muscular atrophy, or age-related macular degeneration. In some embodiments, the pharmaceutical compositions may be used to treat or prevent prostate cancer. In some embodiments, the pharmaceutical compositions may be used to treat or prevent one or more of castration-resistant prostate cancer, metastatic castration-resistant prostate cancer, castration-recurrent prostate cancer, high-risk castration-sensitive prostate cancer, metastatic high-risk castration-sensitive prostate cancer, hormone-resistant prostate cancer, hormone-refractory prostate cancer, androgen-independent prostate cancer, androgen deprivation resistant prostate cancer, androgen ablation resistant prostate cancer, androgen depletion-independent prostate cancer, anti-androgen-recurrent prostate cancer, metastatic castration-resistant prostate cancer in patients who have already received prior chemotherapy containing docetaxel, newly diagnosed high risk metastatic hormone sensitive prostate cancer (mHSPC), metastatic castration resistant prostate cancer in patients who are asymptomatic, mildly symptomatic after failure of androgen deprivation therapy in whom chemotherapy is not yet clinically indicated, metastatic castration resistant prostate cancer in patients whose disease has progressed on or after a docetaxel-based chemotherapy regimen. In some embodiments, pharmaceutical compositions described herein include a lipophilic API, a long chain fatty acid or a long chain fatty acid glyceride, a phospholipid, and optionally, an antioxidant. In some embodiments the API is abiraterone or a pharmaceutically acceptable salt or ester thereof. In some embodiments, the pharmaceutically acceptable ester of abiraterone is abiraterone acetate. In some embodiments, the API is one listed in Table 1 or Table 2, or a pharmaceutically acceptable salt thereof.

In some embodiments, pharmaceutical compositions described herein can be used in combination therapy with at least one other therapeutic agent. The pharmaceutical composition and the therapeutic agent can act additively or, more preferably, synergistically. In some embodiments, the pharmaceutical composition is administered concurrently with the administration of another therapeutic agent. In some embodiments, a pharmaceutical composition is administered prior or subsequent to administration of another therapeutic agent. In some embodiments, a pharmaceutical composition described herein is administered in combination with a glucocorticoid. In some embodiments, a pharmaceutical composition is administered in combination with prednisone, methyl prednisone, prednisolone or dexamethasone. In some embodiments, pharmaceutical compositions described herein include a lipophilic API, a long chain fatty acid or a long chain fatty acid glyceride, a phospholipid, and optionally, an antioxidant. In some embodiments the API is abiraterone or a pharmaceutically acceptable salt thereof. In some embodiments, the pharmaceutically acceptable salt of abiraterone is abiraterone acetate. In some embodiments, the API is one listed in Table 1 or Table 2, or a pharmaceutically acceptable salt thereof.

### EXAMPLES

### Example 1: Solubility of Abiraterone Acetate in Aqueous Media

Abiraterone acetate was added to various aqueous media (2-5 mL) in a 10 mL glass vial, at 25° C., with stirring. When no further dissolution was observable, the suspension was stirred for a further 24 hours at 25° C. If it appeared the abiraterone acetate was still dissolving, the suspension was stirred for a further 24 hours at 25° C. This cycle was repeated until no further dissolution was observable. The suspension was then centrifuged and the supernatant filtered by microfiltration. The filtrate was diluted and abiraterone acetate content determined by HPLC. The solubility was recorded and is presented in Table 6.

TABLE 6

| | Solubility |
|---|---|
| Solvent | (mg/mL) |
| pH 3.0 buffer | <0.001 |
| pH 4.0 buffer | <0.001 |
| pH 5.0 buffer | <0.001 |
| pH 6.0 buffer | <0.001 |
| pH 7.4 buffer | <0.001 |
| FDA Dissolution medium: | 0.7 |
| 0.25% SLS in 56.5 mM phosphate buffer, pH 4.5 | |

### Example 2: Solubility of Abiraterone Acetate in Lipids (Glycerides)

Abiraterone acetate was added to various lipids (2-5 mL) in a 10 mL glass vial, at 25° C., with stirring. When no further dissolution was observable, the suspension was stirred for a further 24 hours at 25° C. If it appeared the abiraterone acetate was still dissolving, the suspension was stirred for a further 24 hours at 25° C. This cycle was repeated until no further dissolution was observable. The suspension was then centrifuged and the supernatant filtered by microfiltration. The filtrate was diluted and abiraterone acetate content determined by HPLC. The solubility was recorded and is presented in Table 7.

TABLE 7

| | Solubility |
|---|---|
| Solvent | (mg/mL) |
| Capryl monoglyceride | 139.8 |
| Glycerol mono/di-caprylocaprate | 147.0 |
| Propylene Glycol Monocaprylate | 160.0 |
| Isopropyl myristate | 41.0 |
| Glyceryl monooleate | 37.1 |
| Polyglycerol fatty acid ester | 10.0 |
| Corn oil | 31.4 |
| Coconut oil | 30.6 |
| Castor oil | 59.6 |
| Soybean oil | 28.1 |
| Olive oil | 27.3 |

### Example 3: Solubility of Abiraterone Acetate in Fatty Acids

Abiraterone acetate was added to various fatty acids (2-5 mL) in a 10 mL glass vial, at 25° C., with stirring. When no further dissolution was observable, the suspension was stirred for a further 24 hours at 25° C. If it appeared the abiraterone acetate was still dissolving, the suspension was stirred for a further 24 hours at 25° C. This cycle was repeated until no further dissolution was observable. The suspension was then centrifuged and the supernatant filtered by microfiltration. The filtrate was diluted and abiraterone acetate content determined by HPLC. The solubility recorded and is presented in Table 8.

TABLE 8

| | | Solu- |
|---|---|---|
| | | bility |
| Solvent | Chemical Structure | (mg/mL) |
| Acetic | CH₃COOH | 2386.1 |
| acid | | |
| Propionic | CH₃CH₂COOH | 2209.5 |
| acid | | |
| Butyric | CH₃(CH₂)₂COOOH | 1399.3 |
| acid | | |
| Valeric | CH₃(CH₂)₃COOOH | 1104.8 |
| acid | | |
| n-caproic | CH₃(CH₂)₄COOOH | 1572.6 |
| acid | | |
| Heptanoic | CH₃(CH₂)₅COOOH | 1325.9 |
| acid | | |
| n-caprylic | CH₃(CH₂)₆COOOH | 1214.7 |
| acid | | |
| Nonanoic | CH₃(CH₂)₇COOOH | 1084.1 |
| acid | | |
| Oleic | CH₃(CH₂)₇CH═CH(CH₂)₇COOH | 239.0 |
| acid | | |
| Linoleic | CH₃(CH₂)₄CH═CH(CH₂)CH═CH(CH₂)₇COOH | 182.9 |
| acid | | |

### Example 4: Solubility of Abiraterone Acetate in Surfactants

Abiraterone acetate was added to various aqueous media (2-5 mL) in a 10 mL glass vial, at 25° C., with stirring. When no further dissolution was observable, the suspension was stirred for a further 24 hours at 25° C. If it appeared the abiraterone acetate was still dissolving, the suspension was stirred for a further 24 hours at 25° C. This cycle was repeated until no further dissolution was observable. The suspension was then centrifuged and the supernatant filtered by microfiltration. The filtrate was diluted and abiraterone acetate content determined by HPLC. The solubility was recorded and is presented in Table 9.

TABLE 9

| | Solubility |
|---|---|
| Solvent | (mg/mL) |
| Tween 80 | 7.5 |
| Labrasol | 35.4 |
| Polyoxyethylene 8 stearate | 18.1 |
| Polyethylene Glycol Oleate | 52.7 |
| Polyoxyethylene hydrogenated castor oil | 10.1 |

### Example 5: Solubility of Abiraterone Acetate in Co-Solvents

Abiraterone acetate was added to various aqueous media (2-5 mL) in a 10 mL glass vial, at 25° C., with stirring. When no further dissolution was observable, the suspension was stirred for a further 24 hours at 25° C. If it appeared the abiraterone acetate was still dissolving, the suspension was stirred for a further 24 hours at 25° C. This cycle was repeated until no further dissolution was observable. The suspension was then centrifuged and the supernatant filtered by microfiltration. The filtrate was diluted and abiraterone acetate content determined by HPLC. The solubility was recorded and is presented in Table 10.

TABLE 10

| | Solubility |
|---|---|
| Solvent | (mg/mL) |
| Glycerol | 5.0 |
| Propylene glycol | 5.0 |
| Ethanol | 61.1 |
| PEG400 | 16.5 |
| Transcutol | 99.5 |

### Example 6: Solubility of Abiraterone Acetate in Other Media

Abiraterone acetate was added to various media (2-5 mL) in a 10 mL glass vial, at 25° C., with stirring. When no further dissolution was observable, the suspension was stirred for a further 24 hours at 25° C. If it appeared the abiraterone acetate was still dissolving, the suspension was stirred for a further 24 hours at 25° C. This cycle was repeated until no further dissolution was observable. The suspension was then centrifuged and the supernatant filtered by microfiltration. The filtrate was diluted and abiraterone acetate content determined by HPLC. The solubility was recorded and is presented in Table 11.

TABLE 11

| | Solubility |
|---|---|
| Solvent | (mg/mL) |
| 1-octanol | 114.0 |
| Azone (Laurocapram) | 66.0 |
| Capryl monoglyceride | 139.8 |
| Glycerol mono/di-caprylocaprate | 147.0 |
| Propylene Glycol Monocaprylate | 160.0 |
| Isopropyl myristate | 41.0 |
| Glyceryl monooleate | 37.1 |
| Polyglycerol fatty acid ester | 10.0 |

### Example 7: Compositions Comprising Abiraterone Acetate

Various compositions comprising abiraterone acetate were prepared by dissolving the abiraterone acetate, and optionally other components as indicated, in a fatty acid or fatty acid glyceride, and encapsulating a portion of the resulting solution within capsules.

Compositions comprising Abiraterone acetate were prepared as follows:

Formulation F1: Abiraterone acetate (10 g) was dissolved in oleic acid (70 g) and 800 mg of the resulting mixture enclosed in size 00# capsules.

Formulation F2: Abiraterone acetate (5 g) was dissolving in oleic acid (70 g) and 800 mg of the resulting mixture enclosed in size 00# capsules.

Formulation F3: Abiraterone acetate (10 g) was dissolved in octanoic acid (50 g) and 800 mg of the resulting mixture enclosed in size 00# capsules.

Formulation F4: Abiraterone acetate (10 g) and lecithin (60 g, containing 20-30% phosphatidylcholine, from TCI Co.) were dissolved in oleic acid (30 g) and 1000 mg of the resulting mixture enclosed in size 00# capsules.

Formulation F5: Abiraterone acetate (10 g) and lecithin (30 g, containing 20-30% phosphatidylcholine, from TCI Co.) were dissolved in oleic acid (30 g) and 700 mg of the resulting mixture enclosed in size 00# capsules.

Formulation F6: Abiraterone acetate (10 g) and lecithin (15 g, containing 20-30% phosphatidylcholine, from TCI Co.) were dissolved in oleic acid (30 g) and 550 mg of the resulting mixture enclosed in size 00# capsules.

Formulation F7: Abiraterone acetate (10 g) and lecithin (15 g, containing 20-30% phosphatidylcholine, from Croda Co.) were dissolved in oleic acid (70 g) and 950 mg of the resulting mixture enclosed in size 00# capsules.

Formulation F8: Abiraterone acetate (25 g) and lecithin (300 g, containing 20-30% phosphatidylcholine, from Croda Co.) were dissolved in oleic acid (300 g) and 625 mg of the resulting mixture enclosed in size 00# capsules.

Formulation F9: Abiraterone acetate (5 g) and lecithin (10 g, containing 85-88% phosphatidylcholine, from Croda Co.) were dissolved in glyceryl caprylate/caprate (MCM) (40 g) and 450 mg of the resulting mixture enclosed in size 00# capsules.

Formulation F10: Abiraterone acetate (5 g) and lecithin (15 g, containing 85-88% phosphatidylcholine, from Croda Co.) were dissolved in MCM (35 g) and olive oil (35 g) with stirring to ensure complete dissolution and 550 mg of the resulting mixture enclosed in size 00# capsules.

Formulation F11: Abiraterone acetate (5 g) and lecithin (10 g, containing 85-88% phosphatidylcholine, from Croda Co.) were dissolved in castor oil (100 g) and 115 mg of the resulting mixture enclosed in size 00# capsules.

Formulation F12: Abiraterone acetate (5 g) and lecithin (10 g, containing 85-88% phosphatidylcholine, from Croda Co.) were dissolved in castor oil (25 g) and MCM (25 g) and 65 mg of the resulting mixture enclosed in size 00# capsules.

Formulation F13: Abiraterone acetate (5 g) and lecithin (15 g, containing 85-88% phosphatidylcholine, from Croda Co.) were dissolved in olive oil (35 g) and MCM (35 g), and enclosing in 00# capsules, each capsule contains 50 mg abiraterone acetate.

Formulation F14: Oleic acid (10.5 g), labrasol (8.4 g), Tween 80 (19.6 g) and Transcutol (31.5 g) were mixed together to provide a clear solution. Abiraterone acetate (10 g) was dissolved in the solution and 800 mg of the resulting mixture enclosed in size 00# capsules. Portions of this formulation were added to distilled water (mass ratio 1:10, 1:100, or 1:1000) without stirring, to rapidly form a milky emulsion which was stable for long periods of time, without precipitation or two phase separation.

Formulation F15: Abiraterone acetate (5 g) was dissolved in a mixture of oleic acid (69 g) and ethanol (2.8 g). Poloxamer 407 (2 g) was dissolved in water (1.2 g). The two solutions were mixed to provide a clear solution and 800 mg of the resulting mixture enclosed in size 00# capsules.

Formulation F16: Abiraterone acetate (5 g) was dissolved in a mixture of oleic acid (67 g) and ethanol (2.8 g). Poloxamer 407 (4 g) was dissolved in water (1.2 g). The two solutions were mixed to provide a clear solution and 800 mg of the resulting mixture enclosed in size 00# capsules.

Formulation F17: Abiraterone acetate (10 g) and lecithin (15 g, containing 85-88% phosphatidylcholine) were dissolved in oleic acid (40 g) and 650 mg of the resulting mixture enclosed in size 00# capsules.

Formulation F18: Abiraterone acetate (10 g) and lecithin (30 g, containing 85-88% phosphatidylcholine) were dissolved in oleic acid (70 g) and 1100 mg of the resulting mixture enclosed in size 00# capsules.

The various components per unit dose for formulations F1-F18 are presented in Table 12.

TABLE 12

| | Amount | | Amount | | | |
|---|---|---|---|---|---|---|
| | Abiraterone | | Oleic | Amount | | Total |
| | acetate | | Acid | Lecithin | Other components | Weight |
| F | (mg) | | (mg) | (mg) | (amount in mg) | (mg) |
| F1 | 100 | mg | 700 mg | | | 800 |
| F2 | 50 | mg | 700 mg | | | 750 |
| F3 | 50 | mg | | | Octanoic acid | 800 |
| | | | | | (750 mg) | |
| F4 | 100 | mg | 300 mg | 600 mg | | 1000 |
| F5 | 100 | mg | 300 mg | 300 mg | | 700 |
| F6 | 100 | mg | 300 mg | 150 mg | | 550 |
| F7 | 100 | mg | 700 mg | 150 mg | | 950 |
| F8 | 25 | mg | 300 mg | 300 mg | | 625 |
| F9 | 50 | mg | | 100 mg | MCM (400 mg) | 550 |
| F10 | 50 | mg | 350 mg | 150 mg | MCM (350 mg) | 900 |
| F11 | 50 | mg | | 100 mg | Castor Oil | 1150 |
| | | | | | (1000 mg) | |
| F12 | 50 | mg | | 100 mg | Castor Oil (250 mg) | 650 |
| | | | | | MCM (250 mg) | |
| F13 | 50 | mg | | 150 mg | Olive oil (350 mg) | 900 |
| | | | | | MCM (350 mg) | |
| F14 | 100 | mg | 105 mg | | Labrasol (84 mg) | 520 |
| | | | | | Tween 80 (196 mg) | |
| | | | | | Transcutol (315 mg) | |
| F15 | 50 | mg | 690 mg | | Ethanol (28 mg) | 800 |
| | | | | | Poloxamer (20 mg) | |
| | | | | | Water (12 mg) | |
| F16 | 50 | mg | 670 mg | | Ethanol (28 mg) | 800 |
| | | | | | Poloxamer (40 mg) | |
| | | | | | Water (12 mg) | |
| F17 | 100 | mg | 400 mg | 150 mg | | 650 |
| F18 | 100 | mg | 700 mg | 300 mg | | 1100 |

The following formulations are prepared according to the same procedures as described for formulations F1-F10, in the amounts shown in Table 13.

TABLE 13

| | Abira- | | | | | | |
|---|---|---|---|---|---|---|---|
| Formu- | terone | | leci- | oleic | butylated | | butylated |
| lation | acetate | | thin | acid | hydroxyanisole | | hydroxytoluene |
| F101 | 5 | g | 15 g | 70 g | 0.45 | g (0.5%) | — |
| F102 | 5 | g | 15 g | 70 g | 0.243 | g (0.27%) | — |
| F103 | 5 | g | 15 g | 70 g | 0.09 | g (0.1%) | — |

| | | | | | | | |
|---|---|---|---|---|---|---|---|
| F104 | 5 | g | 15 g | 70 g | — | 0.45 | g (0.5%) |
| F105 | 5 | g | 15 g | 70 g | — | 0.036 | g (0.04%) |
| F106 | 5 | g | 15 g | 70 g | — | 0.09 | g (0.1%) |

| | | | | | | | |
|---|---|---|---|---|---|---|---|
| F107 | 10 | g | 15 g | 70 g | 0.475 | g (0.5%) | — |
| F108 | 10 | g | 15 g | 70 g | 0.257 | g (0.27% | — |
| F109 | 10 | g | 15 g | 70 g | 0.095 | g (0.1%) | — |

| | | | | | | | |
|---|---|---|---|---|---|---|---|
| F110 | 10 | g | 15 g | 70 g | — | 0.475 | g (0.5%) |
| F111 | 10 | g | 15 g | 70 g | — | 0.038 | g (0.04% |
| F112 | 10 | g | 15 g | 70 g | — | 0.095 | g (0.1%) |

| | | | | | | | |
|---|---|---|---|---|---|---|---|
| F113 | 15 | g | 15 g | 70 g | 0.5 | g (0.5%) | — |
| F114 | 15 | g | 15 g | 70 g | 0.27 | g (0.27%) | — |
| F115 | 15 | g | 15 g | 70 g | 0.1 | g (0.1%) | — |

| | | | | | | | |
|---|---|---|---|---|---|---|---|
| F116 | 15 | g | 15 g | 70 g | — | 0.5 | g (0.5%) |
| F117 | 15 | g | 15 g | 70 g | — | 0.04 | g (0.04%) |
| F118 | 15 | g | 15 g | 70 g | — | 0.1 | g (0.1%) |

### Example 8: Pharmacokinetics of ZYTIGA® in Dogs

Pharmacokinetics parameters of ZYTIGA® tablets were administered to six beagle dogs (three male and three female, purchased from Beijing Marshall Biotechnology Co., Ltd; average weight 6 kg; average age 6 months). All the dogs were fasted for 12 hours prior to dosing (food box removed) and drinking water removed 2 hours before and after dosing. Each dog was administered two ZYTIGA® tablets (500 mg abiraterone acetate) orally, with 50 mL water during the administration. This study was repeated in three different occasions.

Blood samples were collected at 0.25, 0.5, 1, 1.5, 2, 3, 4, 8, 12, 24 h after administration. Whole blood was centrifuged (5 min, 8000 rpm), and the plasma transferred to a 96-well plate and stored at −20° C. The concentration of EDTA-2K⁺ anticoagulated canine plasma was measured by LC-MS/MS. Pharmacokinetic parameters were calculated using a non-compartment model (WinNonlin 6.3 software).

AUC₀₋ₜ was calculated by trapezoidal method AUC_{0-∞}=AUC₀₋ₜ+Ct/ke (where Ct=last plasma concentration; ke=clearance rate constant)

The study was performed in triplicate and the results presented in Table 14.

TABLE 14

| | *Fast- | | | | |
|---|---|---|---|---|---|
| | ing | | | | |
| Formu- | Con- | Dose | Tₘₐₓ | Cₘₐₓ | AUC |
| lation | dition | (mg) | (h) | (ng/ml) | (h*ng/ml) |
| ZYTIGA ® | (a) | 500 | 1.75 | 306.9 ± 218.5 | 1198.0 ± 1271.0 |
| ZYTIGA ® | (b) | 500 | 1.25 | 265.4 ± 143.6 | 1183.4 ± 1524.4 |
| ZYTIGA ® | (c) | 500 | 1.67 | 394.8 ± 168.9 | 1258.7 ± 618.9 |
| Mean | | 500 | 1.56 | 322.4 | 1213.4 |

*Fasting Conditions (a), (b) and (c) are the same.

### Example 9: Oral Bioavailability of Formulations F1-F18 in Dogs

The in vivo pharmacokinetic properties (Tₘₐₓ, Cₘₐₓ, AUC) of the abiraterone acetate compositions prepared as described in Example 7 were examined by administering such compositions to beagle dogs, as described below.

Six beagle dogs (three male and three female, purchased from Beijing Marshall Biotechnology Co., Ltd; average weight 6 kg; average age 6 months), were fasted for various amounts of time as follows: (a) 12 hours before administration, and (b) 2 hours before and 2 hours after administration. Test formulations (F1-F18) were orally administered to each dog. Blood samples were collected at 0.25, 0.5, 1, 1.5, 2, 3, 4, 8, 12, 24 hours after administration and the concentrations of abiraterone were determined. From these measurements, Cₘₐₓ, Tₘₐₓ and AUC were calculated and are presented in Table 15. Relative bioavailability of the test formulations as compared to ZYTIGA were calculated as the ratio of AUC of test formulation/AUC of ZYTIGA formulation, adjusted for the difference in doses, and is presented in the final column. In additional experiments, formulations similar to F2, F4, and F7 have a AUC_{F} close to 4-10 folds (data not shown).

TABLE 15

| | | | | | | | AUC_{F} |
|---|---|---|---|---|---|---|---|
| | | Tₘₐₓ | Cₘₐₓ | Cₘₐₓ | AUC | AUC | AUC_{ZYTEGA} |
| Formulation | Dose (mg) | (h) | (ng/mL) | (±SD) | (h*ng/mL) | (±SD) | (dose adjusted) |
| ZYTIGA ® | 500 | 1.75 | 306.9 | 218.5 | 1198.0 | 1271.0 | |
| F1 | 100 | 1 | 711.3 | 329.6 | 1178.4 | 562.6 | 4.9 |
| F2 | 100 | 1 | 1499.0 | 668.4 | 2893.0 | 953.1 | 12.1 |
| F3 | 100 | 1 | 439.8 | 180.1 | 981.4 | 365.1 | 4.1 |
| F4 | 100 | 0.5 | 2202 | 775.2 | 3657.0 | 1135.0 | 15.3 |
| F5 | 100 | 1 | 1042.8 | 646.0 | 2036.9 | 769.0 | 8.5 |
| F6 | 100 | 1 | 781.9 | 423.7 | 1399.2 | 602.3 | 5.8 |
| F7 | 100 | 0.75 | 2407 | 992 | 4395 | 1479 | 18.3 |
| F8 | 25 | 1.25 | 92.2 | 20.7 | 176.0 | 28.5 | 2.9 |
| F9 | 100 | 0.75 | 193.5 | 50.7 | 332.2 | 102.9 | 2.8 |
| F10 | 50 | 0.5 | 825.9 | 423.2 | 1070.1 | 285.7 | 8.8 |
| F11 | 50 | 1 | 298.9 | 173.3 | 522.5 | 216.4 | 4.3 |
| F12 | 50 | 1 | 328.0 | 246.6 | 485.3 | 239.5 | 4.0 |
| F13 | 50 | 0.5 | 825.9 | 423.2 | 1070.1 | 285.7 | 8.8 |
| F14 | 100 | 1 | 1082.8 | 509.1 | 1806.2 | 651.9 | 7.8 |
| F15 | 50 | 1 | 194.5 | 102.5 | 405.5 | 49.0 | 3.4 |
| F16 | 50 | 1 | 323.2 | 164.1 | 510.5 | 185.7 | 4.3 |
| F17 | 100 | 1.5 | 695.5 | 247.6 | 1650.8 | 596.4 | 6.8 |
| F18 | 100 | 1 | 791.5 | 471.5 | 1724.1 | 571.6 | 7.1 |

### Example 10: In Vitro Digestion Assay

In vitro digestion testing is a useful screening tool to identify formulations with desirable in vivo absorption features. The assay examines lipid-based formulations to reveal the drug distribution between the phases of the formulation, i.e. determination of the patterns of drug partitioning between: (i) a solid precipitate (“pellet phase”); (ii) an aqueous micellar phase; and (iii) an undigested/partially digested oil phase (if present). It is generally understood (see for example Feeney et al., “50 years of oral lipid-based formulations provenance, progress and future perspectives.” Advanced Drug Delivery Reviews, 2016, 101, in particular pages 177-178) that maximizing drug concentration in the aqueous phase (and minimizing drug precipitation) is expected to maximize drug absorption from lipid-based formulations in vivo, i.e. formulations that provide good post-digestion solubilization typically provide good in vivo exposure.

In vitro digestion tests were performed as follows for Formulations F1, F3, F4, F5, F6, F7, F14, F18 and F19. See Table 16. Fasted intestinal medium containing tris-maleate (2 mM), calcium chloride (1.4 mM, dihydrate), sodium chloride (150 mM), sodium taurodeoxycholate (3 mM) and phosphatidylcholine (0.75 mM) was prepared. Formulation, as described herein, (1 g) was dispersed in fasted intestinal medium (40 mL) at 37° C. using a pH-stat titrator (see LFCS Consortium; Williams, H D. et al., J. Pharm. Sci. 101 (2012), p. 3360-3380). Digestion was initiated by addition of porcine pancreatic extract (4 mL) and the pH maintained at 6.5 with sodium hydroxide (0.2M or 0.6M) for 60 min. Aliquots were removed and centrifuged to produce a multiphase sample consisting of: a pellet phase, containing any precipitated abiraterone; an aqueous phase, containing abiraterone in free solution and solubilized in small micelles and vesicles; and, if present, a lipid phase containing abiraterone solubilized in undigested oil droplets and/or larger colloids.

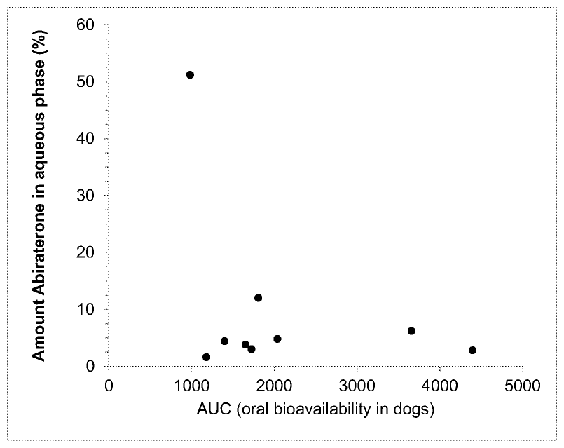

Abiraterone concentrations in each phase were measured by HPLC and the amounts in each phase are described in Table 16. Also described in Table 16 are the AUC results for the relevant formulations, from the oral bioavailability study in dogs (see Example 4). FIG. 1 provides a graphical plot of in vivo oral bioavailability (as AUC) versus amount of abiraterone in the aqueous phase, demonstrating poor correlation between the two measurements and showing that the formulations provided herein show surprisingly good in vivo bioavailability given the in vitro prediction data.

TABLE 16

| Formulation Details | | | | | Assay Results | | |
|---|---|---|---|---|---|---|---|
| | | Oleic | | | Non-aq | | |
| | AA | Acid | Lecithin | Other | Phase | Aq Phase | AUC |
| F1 | 100 mg | 700 mg | — | | 98.4% | 1.6% | 1178 |
| F3 | 100 mg | — | — | Octanoic acid | 48.8% | 51.2% | 981 |
| | | | | (1400 mg) | | | |
| F4 | 100 mg | 300 mg | 600 mg | | 93.8% | 6.2% | 3657 |
| F5 | 100 mg | 300 mg | 300 mg | | 95.2% | 4.8% | 2037 |
| F6 | 100 mg | 300 mg | 150 mg | | 95.6% | 4.4% | 1399 |
| F7 | 100 mg | 700 mg | 150 mg | | 97.2% | 2.8% | 4395 |
| F14 | 100 mg | 105 mg | — | Labrasol (84 mg) | 88.0% | 12.0% | 1806 |
| | | | | Tween 80 (196 mg) | | | |
| | | | | Transcutol (315 mg) | | | |
| F18 | 100 mg | 400 mg | 150 mg | | 96.2% | 3.8% | 1651 |
| F19 | 100 mg | 700 mg | 300 mg | | 97.0% | 3.0% | 1724.1 |
| — | 100 mg | 700 mg | 70 mg | | 97.0% | 3.0% | NA |
| — | 100 mg | 700 mg | 117 mg | | 97.4% | 2.6% | NA |
| — | 100 mg | 700 mg | 350 mg | | 96.6% | 3.4% | NA |

### Example 11: Pharmaceutical Compositions

Compositions described in Table 17 are prepared using methods generally described herein to include the following ingredients.

TABLE 17

| No. | API | Fatty acid | surfactant |
|---|---|---|---|
| 1 | Abiraterone acetate | oleic acid | lecithin |
| 2 | Nilotinib | oleic acid | lecithin |
| 3 | Chlordiazepoxide | oleic acid | lecithin |
| 4 | Naringenin | oleic acid | lecithin |
| 5 | Clofibric acid | oleic acid | lecithin |
| 6 | Hesperetin | oleic acid | lecithin |
| 7 | Cinchonidine | oleic acid | lecithin |
| 8 | Quinine | oleic acid | lecithin |
| 9 | Tramadol | oleic acid | lecithin |
| 10 | Lormetazepam | oleic acid | lecithin |
| 11 | Trazodone | oleic acid | lecithin |
| 12 | Diltiazem | oleic acid | lecithin |
| 13 | Brompheniramine | oleic acid | lecithin |
| 14 | Alprenolol | oleic acid | lecithin |
| 15 | Propranolol | oleic acid | lecithin |
| 16 | Diazepam | oleic acid | lecithin |
| 17 | Apigenin | oleic acid | lecithin |
| 18 | Venlafaxine | oleic acid | lecithin |
| 19 | Rosiglitazone | oleic acid | lecithin |
| 20 | Pyrilamine | oleic acid | lecithin |
| 21 | Quetiapine | oleic acid | lecithin |
| 22 | Ketoprofen | oleic acid | lecithin |
| 23 | Chlorphenamine | oleic acid | lecithin |
| 24 | Fenbufen | oleic acid | lecithin |
| 25 | Naproxen | oleic acid | lecithin |
| 26 | Warfarin | oleic acid | lecithin |
| 27 | Diphenhydramine | oleic acid | lecithin |
| 28 | Bupivacaine | oleic acid | lecithin |
| 29 | Clotiazepam | oleic acid | lecithin |
| 30 | Chrysin | oleic acid | lecithin |
| 31 | Valsartan | oleic acid | lecithin |
| 32 | Haloperidol | oleic acid | lecithin |
| 33 | Flurbiprofen | oleic acid | lecithin |
| 34 | Progesterone | oleic acid | lecithin |
| 35 | Celecoxib | oleic acid | lecithin |
| 36 | Flurazepam | oleic acid | lecithin |
| 37 | Glimepiride | oleic acid | lecithin |
| 38 | Ibuprofen | oleic acid | lecithin |
| 39 | Atorvastatin | oleic acid | lecithin |
| 40 | Indometacin | oleic acid | lecithin |
| 41 | Nortriptyline | oleic acid | lecithin |
| 42 | Loratadine | oleic acid | lecithin |
| 43 | Fluoxetine | oleic acid | lecithin |
| 44 | Diclofenac | oleic acid | lecithin |
| 45 | Clopidogrel | oleic acid | lecithin |
| 46 | Duloxetine | oleic acid | lecithin |
| 47 | Penbutolol | oleic acid | lecithin |
| 48 | Maprotiline | oleic acid | lecithin |
| 49 | Imipramine | oleic acid | lecithin |
| 50 | Amitriptyline | oleic acid | lecithin |
| 51 | Cyproheptadine | oleic acid | lecithin |
| 52 | Sertraline | oleic acid | lecithin |
| 53 | Flufenamic acid | oleic acid | lecithin |
| 54 | Chlorpromazine | oleic acid | lecithin |
| 55 | Miconazole | oleic acid | lecithin |
| 56 | Rimonabant | oleic acid | lecithin |
| 57 | Clofazimine | oleic acid | lecithin |
| 58 | Enzalutamide | oleic acid | lecithin |

While particular embodiments described herein have been shown and described herein, such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope and that methods and structures within the scope of these claims and their equivalents be covered thereby.

1. A pharmaceutical composition, comprising:
a) an active pharmaceutical ingredient or a pharmaceutically acceptable salt or ester thereof, wherein the active pharmaceutical ingredient has a calculated log P in octanol-water equal or greater than 4.0;
b) at least one long chain fatty acid or at least one fatty acid glyceride; and
c) a phospholipid.

2. The pharmaceutical composition of claim 1, wherein the active pharmaceutical ingredient has a calculated log P equal or greater than 4.5.

3. The pharmaceutical composition of claim 1, wherein the active pharmaceutical ingredient has a calculated log P equal or greater than 5.

4. The pharmaceutical composition of claim 1, wherein the solubility of the active pharmaceutical ingredient in the at least one long chain fatty acid or at least one fatty acid glyceride is at least 25 mg/mL.

5. The pharmaceutical composition of claim 1, wherein the phospholipid is a phosphatidylcholine, a phosphatidylethanolamine, a phosphatidylinositol, a phosphatidylserine, a plasmalogen, a sphingomyelins, lecithin or phosphatidic acid

6. The pharmaceutical composition of claim 1, wherein the phospholipid is a component of lecithin.

7. The pharmaceutical composition of claim 6, wherein the lecithin is isolated from egg yolk.

8. A pharmaceutical composition, comprising:
a) an active pharmaceutical ingredient having a weak base function group or a pharmaceutically acceptable salt thereof, wherein the active pharmaceutical ingredient has a calculated log P in octanol-water equal or greater than 4.0, and a pKa equal or greater than 3.0;
b) at least one long chain fatty acid; and
c) a phospholipid.

9. The pharmaceutical composition of claim 8, wherein the at least one long chain fatty acid is oleic acid.

10. The pharmaceutical composition of claim 8, wherein the phospholipid is phosphatidylcholine.

11. The pharmaceutical composition of claim 8, wherein the phospholipid is present in an amount of about 100 mg to about 200 mg.

12. The pharmaceutical composition of claim 8, wherein phospholipid comprises 10%-20% of the total weight of the composition.

13. The pharmaceutical composition of claim 8, wherein phospholipid comprises 14%-17% of the total weight of the composition.

14. The pharmaceutical composition of claim 8, wherein a ratio by weight of the at least one long chain fatty acid to phospholipid is greater than 1.5.

15. The pharmaceutical composition of claim 8, wherein phospholipid comprises greater than 80% phosphatidylcholine.

16. A pharmaceutical composition, comprising:
a) abiraterone or a pharmaceutically acceptable salt or ester thereof;
b) at least one long chain fatty acid or at least one fatty acid glyceride; and
c) lecithin.

17. The pharmaceutical composition of claim 16, wherein the pharmaceutically acceptable ester of the abiraterone is abiraterone acetate.

18. A pharmaceutical composition, comprising:
a) a compound of the formula (I), 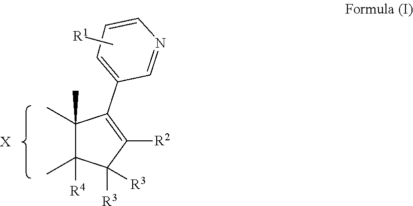
wherein X represents the residue of the A, B and C rings of a steroid selected from the group consisting of
androstan-3α- or 3β-ol,
androst-5-en-3α- or 3β-ol,
androst-4-en-3-one,
androst-2-ene,
androst-4-ene,
androst-5-ene,
androsta-5,7-dien-3α or 3β-ol,
androsta-1,4-dien-3-one,
estra-1,3,5[10]-trien-3-ol,
α-androstan-3-one,
androsta-3,5-diene,
androsta-3,5-diene-3-ol,
estra-1,3,5[10]-triene,
estra-1,3,5[10]-trien-3-ol,
5α-androstan-3-one,
androst-4-ene-3,11-dione,
6-fluoroandrost-4-ene-3-one, and
androstane-4-ene-3,6-dione,
each of which, where structurally permissible, can be further derivatized in one or more of the following ways:
to form 3-esters
to have one or more carbon or carbon ring double bonds in any of the 5,6-, 6,7-, 7,8-, 9,11- and 11,12-positions
as 3-oximes
as 3-methylenes
as 3-carboxylates
as 3-nitriles
as 3-nitros
as 3-desoxy derivatives
to have one or more hydroxy, halo, C₁₋₄-alkyl, trifluoro-methyl, C₁₋₄-alkoxy, C₁₋₄-alkanoyloxy, benzoyloxy, oxo, methylene or alkenyl substituents in the A, B, or C-ring,
to be 19-nor;
R¹ represents a hydrogen atom or an alkyl group of 1-4 carbon atoms;
R⁴ represents a hydrogen atom, a halogen atom or an alkyl group of 1 to 4 carbon atoms;
each of the R³ substituents independently represents a hydrogen atom or an alkyl or alkoxy group of 1-4 carbon atoms, a hydroxy group or an alkylcarbonyloxy group of 2 to 5 carbon atoms or together represent an oxo or methylene group, or R⁴ and one of the R³ groups together represent a double bond and the other R³ group represents a hydrogen atom or an alkyl group of 1 to 4 carbon atoms; and
R² represents a hydrogen atom, halogen atom, or an alkyl group of 1 to 4 carbon atoms, in the form of the free bases or pharmaceutically acceptable acid addition salts;
b) at least one long chain fatty acid or at least one fatty acid glyceride; and
c) lecithin.

19. The pharmaceutical composition of claim 18, wherein the compound is saturated and unsubstituted at the 11- and 12-positions.

20. The pharmaceutical composition of claim 18, wherein the compound is selected from:
17-(3-pyridyl)androsta-5,16-dien-3β-ol,
17-(3-pyridyl)androsta-3,5,16-triene,
17-(3-pyridyl)androsta-4,16-dien-3-one,
17-(3-pyridyl)estra-1,3,5[10], 16-tetraen-3-ol,
17-(3-pyridyl)-5α-androst-16-en-3α-ol,
17-(3-pyridyl)-5α-androst-16-en-3-one,
17-(3-pyridyl)-androsta-4,16-diene-3,11-dione,
17-(3-pyridyl)-androsta-3,5,16-trien-3-ol,
6α- and 6β-fluoro-17-(3-pyridyl)androsta-4,16-dien-3-one,
17-(3-pyridyl)androsta-4,16-dien-3,6-dione,
3α-trifluoromethyl-17-(3-pyridyl)androst-16-en-3β-ol,
and their acid addition salts and 3-esters.

21. The pharmaceutical composition of claim 18, wherein R¹ is hydrogen.

22. The pharmaceutical composition of claim 18, wherein the compound is abiraterone acetate.

23. The pharmaceutical composition of claim 17 or 22, wherein the abiraterone acetate is present in an amount of from 25 mg to 200 mg.

24. The pharmaceutical composition of claim 17 or 22, wherein the abiraterone acetate is present in an amount of about 50 mg, about 100 mg or about 150 mg.

25. The pharmaceutical composition of claim 16 or 18, wherein the at least one long chain fatty acid or the at least one fatty acid glyceride comprises greater than 11 carbon atoms.

26. The pharmaceutical composition of claim 16 or 18, wherein the at least one long chain fatty acid or the at least one fatty acid glyceride consists of 12, 14, 16, 18, 20, 22 or 24 carbon atoms.

27. The pharmaceutical composition of claim 16 or 18, wherein the at least one long chain fatty acid glyceride comprises one type of fatty acid glyceride.

28. The pharmaceutical composition of claim 16 or 18, wherein the at least one long chain fatty acid glyceride comprises at least two different fatty acid glycerides.

29. The pharmaceutical composition of claim 16 or 18, wherein the at least one long chain fatty acid comprises one type of long chain fatty acid.

30. The pharmaceutical composition of claim 16 or 18, wherein the at least one long chain fatty acid comprises at least two different long chain fatty acids.

31. The pharmaceutical composition of claim 16 or 18, wherein the at least one long chain fatty acid is unsaturated.

32. The pharmaceutical composition of claim 16 or 18, wherein the at least one long chain fatty acid is saturated.

33. The pharmaceutical composition of claim 16 or 18, wherein the at least one long chain fatty acid is Lauric acid, Myristic acid, Palmitic acid, Stearic acid, Arachidic acid, Behenic acid, Lignoceric acid or Cerotic acid.

34. The pharmaceutical composition of claim 16 or 18, wherein the at least one long chain fatty acid is Myristoleic acid, Palmitoleic acid, Sapienic acid, Oleic acid, Elaidic acid, Vaccenic acid, Linoleic acid, Linolelaidic acid, γ-Linolenic acid, α-Linolenic acid, Stearidonic acid, Paullinic acid, Gondoic acid, Dihomo-γ-linolenic acid, Mead acid, Arachidonic acid, Eicosapentaenoic acid, Erucic acid, Docosatetraenoic acid, Docosahexaenoic acid or Nervonic acid.

35. The pharmaceutical composition of claim 16 or 18, wherein the at least one long chain fatty acid or the at least one fatty acid glyceride is present in an amount of 100 mg to about 1000 mg.

36. The pharmaceutical composition of claim 16 or 18, wherein the at least one long chain fatty acid or the at least one fatty acid glyceride is present in an amount of about 700 mg.

37. The pharmaceutical composition of claim 16 or 18, wherein the at least one long chain fatty acid or the at least one fatty acid glyceride comprises 30%-95% of the total weight of the composition.

38. The pharmaceutical composition of claim 16 or 18, wherein the at least one long chain fatty acid or the at least one fatty acid glyceride comprises 60%-80% of the total weight of the composition.

39. The pharmaceutical composition of claim 16 or 18, wherein a ratio of the at least one long chain fatty acid or at least one fatty acid glyceride to the lecithin is greater than 1.5.

40. The pharmaceutical composition of claim 16 or 18, wherein the lecithin is present in an amount of from 100 mg to 200 mg.

41. The pharmaceutical composition of claim 16 or 18, wherein the at least one long chain fatty acid or at least one fatty acid glyceride is oleic acid.

42. The pharmaceutical composition of claim 41, wherein the oleic acid is present in an amount of from 600 mg to 800 mg.

43. The pharmaceutical composition of claim 17 or 22, wherein the abiraterone acetate, the at least one long chain fatty acid or at least one fatty acid glyceride, and the lecithin are present in a ratio by weight of 1:(4.66 to 14):(1 to 3), wherein the at least one long chain fatty acid or at least one fatty acid glyceride is oleic acid.

44. The pharmaceutical composition of claim 17 or 22, wherein the abiraterone acetate, the at least one long chain fatty acid or at least one fatty acid glyceride, and the lecithin are present in a ratio by weight of about 1:14:3, about 1:7:1.5, or about 1:4.66:1, wherein the at least one long chain fatty acid or at least one fatty acid glyceride is oleic acid.

45. The pharmaceutical composition of any one of the preceding claims, further comprising an antioxidant.

46. The pharmaceutical composition of claim 45, wherein the antioxidant is vitamin E, butylated hydroxytoluene, butylated hydroxyanisole, ascorbyl palmitate, terbuteryl hydroquinone or a combination thereof.

47. The pharmaceutical composition of claim 45, wherein the antioxidant is butylated hydroxyanisole or butylated hydroxytoluene or a combination thereof.

48. The pharmaceutical composition of claim 45, wherein the antioxidant is present in an amount less than 1.5% of the total weight of the composition.

49. The pharmaceutical composition of any one of the preceding claims, wherein the pharmaceutical composition is formulated for oral administration.

50. The pharmaceutical composition of any one of the preceding claims, wherein the pharmaceutical composition is formulated for once daily dosing.

51. The pharmaceutical composition of any one of the preceding claims, wherein the pharmaceutical composition is formulated for twice daily dosing.

52. A method for modulating androgen receptor activity, comprising administering to a subject in need of modulating androgen receptor activity, the pharmaceutical composition of any one of claims 1 to 51.

53. A method for treating prostate cancer, breast cancer, ovarian cancer, endometrial cancer, bladder cancer, pancreatic cancer, hepatocellular cancer, kidney cancer, liver cancer, salivary gland carcinoma, hair loss, acne, hirsutism, ovarian cysts, polycystic ovary disease, precocious puberty,
spinal and bulbar muscular atrophy, or age-related macular degeneration, comprising administering to a subject in need thereof the pharmaceutical composition of any one of claims 1 to 51.

54. A method for treating prostate cancer, comprising administering to a subject in need thereof the pharmaceutical composition of any one of claims 1 to 51.

55. The method of claim 54, wherein the prostate cancer is selected from one or more of the following:
castration-resistant prostate cancer;
metastatic castration-resistant prostate cancer;
castration-recurrent prostate cancer
high-risk castration-sensitive prostate cancer;
metastatic high-risk castration-sensitive prostate cancer;
hormone-resistant prostate cancer;
hormone-refractory prostate cancer;
androgen-independent prostate cancer;
androgen deprivation resistant prostate cancer;
androgen ablation resistant prostate cancer;
androgen depletion-independent prostate cancer;
anti-androgen-recurrent prostate cancer;
metastatic castration-resistant prostate cancer in patients who have already received prior chemotherapy containing docetaxel;
newly diagnosed high risk metastatic hormone sensitive prostate cancer (mHSPC);
metastatic castration resistant prostate cancer in patients who are asymptomatic or mildly symptomatic after failure of androgen deprivation therapy in whom chemotherapy is not yet clinically indicated; and
metastatic castration resistant prostate cancer in patients whose disease has progressed on or after a docetaxel-based chemotherapy regimen.

56. The method of any one of claims 52 to 55, further comprising administering a glucocorticoid.

57. The method of any one of claims 52 to 55, further comprising administering prednisone, methyl prednisone, prednisolone or dexamethasone.

58. The method of any one of claims 52 to 55, further comprising administering a chemotherapeutic agent.

59. A method for treating prostate cancer, comprising administering to a subject in need thereof a pharmaceutical composition, comprising
a) about 50 mg to about 250 mg of abiraterone acetate;
b) at least one long chain fatty acid or at least one fatty acid glyceride;
c) a surfactant; and
d) optionally an antioxidant,
optionally, wherein a total daily dose of abiraterone acetate administered to the subject is less than about 1000 mg.

60. The method of claim 59, wherein the total daily dose of abiraterone acetate administered to the subject is less than about 500 mg.

61. The method of claim 59, wherein the total daily dose of abiraterone acetate administered to the subject is less than about 250 mg.

62. A method for preparing a pharmaceutical composition, comprising the steps:
(a) combining an active pharmaceutical ingredient, a surfactant, and, optionally, an antioxidant;
(b) dissolving the mixture of step (a) in a long chain fatty acid or fatty acid glyceride;
(c) enclosing the mixture of step (b) within a capsule.

63. Use of the pharmaceutical composition of any of claims 1 to 51 in preparation of a medicament for modulating androgen receptor activity or treating prostate cancer.

64. A kit, comprising the pharmaceutical composition of any of claims 1 to 51.

65. The pharmaceutical composition of any of claims 1 to 51 formulated into a form of capsule.

66. A pharmaceutical composition, comprising:
a) abiraterone or a pharmaceutically acceptable salt or ester thereof; and
b) at least one long chain fatty acid, preferably oleic acid.
